(19) United States
(12) Patent Application Publication
(10) Pub. No.: US 2024/0335135 A1
(43) Pub. Date: Oct. 10, 2024

(54) Breathing Signal Identification Using Radio Signals

(71) Applicant: Emerald Innovations Inc., Cambridge, MA (US)

(72) Inventors: Yuqing Ai, Seattle, WA (US); Rumen Hristov, Cambridge, MA (US); Dina Katabi, Boston, MA (US); Bruce Maggs, Chapel Hill, NC (US); Hariharan Rahul, Boston, MA (US); Shichao Yue, Somerville, MA (US)

(21) Appl. No.: 18/629,009

(22) Filed: Apr. 8, 2024

Related U.S. Application Data
(60) Provisional application No. 63/494,823, filed on Apr. 7, 2023.

(51) Int. Cl. A61B 5/08; A61B 5/05

(52) U.S. Cl. CPC A61B 5/0816; A61B 5/05

(57) ABSTRACT

Breathing signals are extracted from reflected wireless signals that are reflected from objects and/or people in a room. Feature maps are formed for the breathing signals at respective time intervals, each feature map representing a correlation of the breathing signals at each voxel in a plurality of voxels that represent three-dimensional locations within the room. The identity of the source of the breathing signals can be determined by statistically comparing the features maps with one or more location templates. Alternatively, at least some of the feature maps can be grouped into clusters. The distance between a respective cluster and a predetermined sleep area of at least one person can be used to identify the source of the breathing signals associated with the respective cluster.

### CROSS-REFERENCE TO RELATED APPLICATIONS

This application claims priority to U.S. Provisional Application No. 63/494,823, titled “Breathing Signal Identification Using Radio Signals,” filed on Apr. 7, 2023, which is hereby incorporated by reference.

### TECHNICAL FIELD

This application relates generally to breathing signal identification using wireless signals.

### BACKGROUND

Radio signals can be used to track the movement of a person in a room and to determine certain physiological information about the person including his/her breathing rate. It is straightforward to identify the source of breathing signals when the room is always occupied by the same person. It is challenging to automatically identify the source of breathing signals when a room may be occupied simultaneously and/or serially by two people, as is the case for many couples.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages, and novel features of the disclosure will be set forth in the following detailed description of the disclosure when considered in conjunction with the drawings, which are intended to illustrate, not limit, the invention.

An aspect of the invention is directed to a system for wireless detection of breathing from multiple people, comprising: one or more transmitting antennas configured to produce transmitted wireless signals over a time period; one or more transmitting one or more receiving antennas configured to receive reflected wireless signals over the time period, the reflected wireless signals being reflected from objects and/or people in a room; and a computer in electrical communication with the one or more receiving antennas to receive the reflected wireless signals, the computer including a processor and non-transitory computer memory in electrical communication with the processor. The non-transitory computer memory stores computer-readable instructions that, when executed by the processor, cause the processor to: automatically extract breathing signals from the reflected wireless signals; automatically form feature maps for the breathing signals, each feature map representing a correlation of the breathing signals at each voxel in a plurality of voxels that represent three-dimensional locations within the room, each feature map representing a respective time interval of the time period, the time period subdivided into time intervals; automatically statistically compare the features maps with one or more location templates, each location template including a respective correlation of collected breathing signals for a respective known person at each voxel; and automatically identify the breathing signals for a first person and the breathing signals for a second person based, at least in part, on statistical correlations between each location template and each feature map.

In one or more embodiments, the one or more location templates comprise a first location template that includes a first correlation of the collected breathing signals for the first person at each voxel, and a second location template that includes a second correlation of the collected breathing signals for a second person at each voxel, and the computer-readable instructions further cause the processor to: automatically assign, for each feature map having a higher statistical correlation with the first location template compared to with the second location template, the breathing signals associated with a respective feature map to the first person; and automatically assign, for each feature map having a lower statistical correlation with the first location template compared to with the second location template, the breathing signals associated with the respective feature map to the second person.

In one or more embodiments, the statistical correlations include Pearson correlations. In one or more embodiments, the computer-readable instructions further cause the processor to: automatically calculate a respective average breathing rate of the breathing signals associated with each feature map; and automatically statistically compare the respective average breathing rate to a respective average location-template breathing rate for each location template. In one or more embodiments, the statistical correlations include: a first statistical comparison of the respective average breathing rate for each feature map with a first average location-template breathing rate of the first location template, and a second statistical comparison of the respective average breathing rate for each feature map with a second average location-template breathing rate of the second location template.

In one or more embodiments, the one or more location templates comprise a first location template that includes a first correlation of the collected breathing signals for the first person at each voxel, and the computer-readable instructions further cause the processor to automatically assign, for each feature map having a respective statistical correlation with the first location template higher than a threshold value, the breathing signals associated with a respective feature map to the first person; and automatically assign, for each feature map having the respective statistical correlation with the first location template lower than or equal to the threshold value, the breathing signals associated with the respective feature map to the second person.

In one or more embodiments, the computer-readable instructions further cause the processor to automatically group at least some of the feature maps into clusters, each cluster representing a group of correlated breathing signals; and automatically identify the breathing signals for the first person and the breathing signals for a second person based, at least in part, on statistical correlations between each location template and the feature maps in each cluster.

In one or more embodiments, the time period is a second time period, the one or more transmitting antennas are configured to produce first transmitted wireless signals over a first time period, the first time period before the second time period, the one or more receiving antennas are configured to receive first reflected wireless signals over the first time period, the first reflected wireless signals being reflected from the objects and/or the people in the room, and the computer-readable instructions further cause the processor to automatically extract the collected breathing signals, with the computer, from the first reflected wireless signals; automatically form first feature maps for the first breathing signals, each first feature map representing a first correlation of the first breathing signals at each voxel, each first feature map representing a respective first time interval of the first time period, the first time period subdivided into first time intervals; automatically group at least some of the first feature maps into one or more clusters, each cluster representing a group of correlated first breathing signals; automatically form the one or more location templates using the one or more clusters; and label each location template with the respective known person.

Another aspect of the invention is directed to a system for wireless detection of breathing from multiple people, comprising: one or more transmitting antennas configured to produce transmitted wireless signals over a time period that includes multiple days; one or more transmitting one or more receiving antennas configured to receive reflected wireless signals over the time period, the reflected wireless signals being reflected from objects and/or people in a room; and a computer in electrical communication with the one or more receiving antennas to receive the reflected wireless signals, the computer including a processor and non-transitory computer memory in electrical communication with the processor. The non-transitory computer memory stores computer-readable instructions that, when executed by the processor, cause the processor to a. automatically extract breathing signals from the reflected wireless signals; b. automatically form feature maps for the breathing signals, each feature map representing a correlation of the breathing signals at each voxel in a plurality of voxels that represent three-dimensional locations within the room, each feature map representing a respective time interval of the time period, the time period subdivided into time intervals; c. automatically group at least some of the feature maps into daily clusters, each daily cluster representing a group of correlated breathing signals for one of the days of the time period; d. automatically identify a plurality of anchor clusters, from the daily clusters, having respective groups of the correlated breathing signals that mutually overlap in time; e. automatically form a plurality of meta clusters, each meta cluster including a respective anchor cluster, wherein the daily clusters not including the anchor clusters comprise non-anchor daily clusters; f. automatically add at least some of the non-anchor daily clusters to the meta clusters according to first statistical correlations between each non-anchor daily cluster and each meta cluster; g. automatically merge at least some of the meta clusters to form merged meta clusters according to second statistical correlations between the meta clusters; h. automatically determine respective distances between each merged meta cluster and one or more predetermined sleep areas of one or more respective people in the room; i. automatically assign the breathing signals associated with a first merged meta cluster to a first person based, at least in part, on one or more first respective distances between the first merged meta cluster and each predetermined sleep area; and j. automatically assign the breathing signals associated with a second merged meta cluster to a second person based, at least in part, on one or more second respective distances between the second merged meta cluster and each predetermined sleep area.

In one or more embodiments, for each respective non-anchor daily cluster not added to one of the meta clusters in step f, a respective new meta cluster is formed that includes the respective daily cluster. In one or more embodiments, the first statistical correlations include a Pearson correlation between average feature maps, and/or a difference between average breathing rates. In one or more embodiments, the second statistical correlations include a Pearson correlation between average feature maps, and/or a difference between average breathing rates.

In one or more embodiments, the one or more predetermined sleep areas comprise a first predetermined sleep area for a first person in the room, and the computer-readable instructions further cause the processor to automatically assign the breathing signals associated with the first merged meta cluster to the first person based on a first distance between the first merged meta cluster and the first predetermined sleep area; and automatically assign the breathing signals associated with the second merged meta cluster to the second person based on a second distance between the first merged meta cluster and the first predetermined sleep area, the first distance smaller than the second distance.

In one or more embodiments, the one or more predetermined sleep areas comprise a first predetermined sleep area for a first person in the room, and a second predetermined sleep area for a second person in the room, and the computer-readable instructions further cause the processor to automatically determine first distances between each merged meta cluster and the first predetermined sleep area; automatically determine second distances between each merged meta cluster and the second predetermined sleep area; automatically assign the breathing signals associated with the first merged meta cluster to the first person, the first merged meta cluster having a smallest first distance with respect to the first distances, and automatically assign the breathing signals associated with the second merged meta cluster to the second person, the second merged meta cluster having a smallest second distance with respect to the second distances.

Another aspect of the invention is directed to a system for wireless detection of breathing from multiple people, comprising: one or more transmitting antennas configured to produce transmitted wireless signals over a time period that includes multiple days; one or more transmitting one or more receiving antennas configured to receive reflected wireless signals over the time period, the reflected wireless signals being reflected from objects and/or people in a room; and a computer in electrical communication with the one or more receiving antennas to receive the reflected wireless signals, the computer including a processor and non-transitory computer memory in electrical communication with the processor. The non-transitory computer memory stores computer-readable instructions that, when executed by the processor, cause the processor to a. automatically extract breathing signals from the reflected wireless signals; b. automatically form feature maps for the breathing signals, each feature map representing a correlation of the breathing signals at each voxel in a plurality of voxels that represent three-dimensional locations within the room, each feature map representing a respective time interval of the time period, the time period subdivided into time intervals; c. automatically group at least some of the feature maps into daily clusters, each daily cluster representing a group of correlated breathing signals for one of the days of the time period; d. automatically group the daily clusters for a respective day into one or more daily meta clusters according to first statistical correlations between the daily clusters for the respective day; c. automatically form first and second all-days meta clusters, the first all-days meta cluster including a first daily meta cluster, the second all-days meta cluster including a second daily meta cluster; f. automatically group each daily meta cluster for each day into the first all-days meta cluster or the second all-days meta cluster, the grouping determined based, at least in part, on second statistical correlations between each daily meta cluster and each all-days meta cluster; g. automatically determine a respective distance between a respective in-bed center location of each all-days meta cluster and one or more predetermined sides of a bed area of one or more respective people in the room; h. automatically assign the breathing signals associated with the first all-days meta cluster to a first person based, at least in part, on one or more first respective distances between a first in-bed center location of the first all-days meta cluster and each predetermined side of the bed area; and i. automatically assign the breathing signals associated with the second all-days meta cluster to a second person based, at least in part, on one or more first respective distances between a second in-bed center location of the second all-days meta cluster and each predetermined side of the bed area.

In one or more embodiments, the one or more predetermined sides of the bed area comprises a first predetermined side of the bed area for the first person, and the computer-readable instructions further cause the processor to automatically assign the breathing signals associated with the first all-days meta cluster to the first person based, at least in part, on a first distance between the first in-bed center location and the first predetermined side of the bed area; and automatically assign the breathing signals associated with the second all-days meta cluster to the second person based, at least in part, on a second distance between the second in-bed center location and the first predetermined side of the bed area, the first distance smaller than the second distance.

In one or more embodiments, the one or more predetermined sides of the bed area comprises a first predetermined side of the bed area for the first person, and a second predetermined side of the bed area for the second person, and the computer-readable instructions further cause the processor to automatically determine first distances between each in-bed center location and the first predetermined sleep area; automatically determine second distances between each in-bed center location and the second predetermined sleep area; automatically assign the breathing signals associated with the first all-days meta cluster to the first person, the first in-bed center location having a smallest first distance with respect to the first distances, and automatically assign the breathing signals associated with the second all-days meta cluster to the second person, the second in-bed center location having a smallest second distance with respect to the second distances.

In one or more embodiments, the first person is a primary person under observation, in steps h and i, the first all-days meta cluster is assigned to the first person and the second all-days meta cluster is assigned to the second person, and the computer-readable instructions further cause the processor to determine a first duration of the breathing signals associated with the first all-days meta cluster and a second duration of the breathing signals associated with the second all-days meta cluster; and after step i: automatically reassign the first all-days meta cluster to the second person; and automatically reassign the second all-days meta cluster to the first person second, wherein the first duration is greater than twice the second duration.

In one or more embodiments, the daily clusters from a first day are grouped into only the first daily meta cluster for the first day, the first all-days meta cluster is formed with the first daily meta cluster from the first day, and the second all-days meta cluster is formed with the second daily meta cluster from a second day, wherein a respective second statistical correlation between the second daily meta cluster from the second day and the first all-days meta cluster is lower than or equal to a threshold value.

In one or more embodiments, the first all-days meta cluster is formed with the first daily meta cluster from a first day, and the second all-days meta cluster is formed with the second daily meta cluster from the first day.

### BRIEF DESCRIPTION OF THE DRAWINGS

For a fuller understanding of the nature and advantages of the present concepts, reference is made to the detailed description of preferred embodiments and the accompanying drawings.

FIG. 1 is a block diagram of a motion tracking system according to an embodiment.

FIG. 2A is a plot of transmit and receive frequencies with respect to time.

FIG. 2B is a plot of received energy with respect to frequency corresponding to FIG. 2A.

FIG. 3A is a spectrogram for one transmit and antenna pair.

FIG. 3B is the spectrogram of FIG. 3A after background subtraction.

FIG. 3C is a plot of estimates of a first round-trip time corresponding to the spectrogram in FIG. 3B.

FIG. 4 is a block diagram of a motion tracking system according to another embodiment.

FIG. 5 is a block diagram of an example room or other indoor environment according to an embodiment.

FIG. 6 is a flow chart of a method for automatically determining the source of breathing signals according to an embodiment.

FIG. 7 illustrates an example heatmap of accumulated position data according to an embodiment.

FIGS. 8A and 8B illustrate two breathing signals extracted from a heatmap according to an embodiment.

FIG. 9 illustrates an example feature map according to an embodiment.

FIG. 10 illustrates an example of clusters of feature maps.

FIG. 11 illustrates an example of merging clusters in a clustered featured map.

FIG. 12 illustrates a clustered feature map after the clusters in FIG. 11 are merged.

FIG. 13 illustrates manual labelling of clusters using polygons.

FIG. 14 is a flow chart of a method for automatically determining the source of breathing signals from multiple people according to an embodiment.

FIG. 15 is a flow chart of a method for automatically determining the source of breathing signals from multiple people according to another embodiment

FIG. 16 is a flow chart of a method for automatically determining the source of breathing signals from multiple people according to another embodiment.

### DETAILED DESCRIPTION

Embodiments of the technology can automate the process by which breathing signals are identified as belonging to specific individuals. Breathing signals generated by other entities such as pets and non-breathing signals generated by inanimate, but moving, objects such as fans can be identified and distinguished. The technology applies to breathing signals that are derived from measurements of reflections of wireless transmissions.

Two breathing signals recorded/detected at different times can be determined as belonging to the same individual if each signal shows a similar correlation with the wireless signals reflected from individual points in space at the time it is recorded/detected. In particular, given a breathing signal and a collection of contemporaneous wireless signals reflected back from a collection of voxels in space, a feature map correlates the breathing signal with the signal from each of the voxels. A location template represents a correlation of collected breathing signals belonging to a known person. Given a breathing signal of unknown origin, its source can be identified by statistically comparing or correlating its feature map with one or more location templates, and selecting the identity associated with the location template that is the closest match.

In another embodiment, at least some of the feature maps can be grouped into daily clusters. Each daily cluster can represent a group of correlated breathing signals for one of the days of the time period. A plurality of anchor clusters, from the daily clusters, can be identified. The anchor clusters have respective groups of the correlated breathing signals that mutually overlap in time. A plurality of meta clusters, with each meta cluster including a respective anchor cluster, can be formed. The anchor clusters not added to a meta cluster can be referred to as non-anchor daily clusters. At least some of the non-anchor daily clusters can be added to the meta clusters according to first statistical correlations between each non-anchor daily cluster and each meta cluster. At least some of the meta clusters can be merged to form merged meta clusters according to second statistical correlations between the meta clusters. Respective distances between each merged meta cluster and one or more predetermined sleep areas of one or more respective people in the room can be determined. The breathing signals associated with a first merged meta cluster can be assigned to a first person based, at least in part, on one or more first respective distances between the first merged meta cluster and each predetermined sleep area. The breathing signals associated with a second merged meta cluster can be assigned to a second person based, at least in part, on one or more second respective distances between the second merged meta cluster and each predetermined sleep area.

In another embodiment, at least some of the feature maps can be grouped into daily clusters. Each daily cluster can represent a group of correlated breathing signals for one of the days of the time period. The daily clusters for a respective day can be grouped into one or more daily meta clusters according to first statistical correlations between the daily clusters for the respective day. First and second all-days meta clusters can be formed where the first all-days meta cluster includes a first daily meta cluster and the second all-days meta cluster includes a second daily meta cluster. Each daily meta cluster for each day can be grouped into the first all-days meta cluster or the second all-days meta cluster. The grouping can be determined based, at least in part, on second statistical correlations between each daily meta cluster and each all-days meta cluster. A respective distance between a respective in-bed center location of each all-days meta cluster and one or more predetermined sides of a bed area of one or more respective people in the room can be determined. The breathing signals associated with the first all-days meta cluster can be assigned to a first person based, at least in part, on one or more first respective distances between a first in-bed center location of the first all-days meta cluster and each predetermined side of the bed area. The breathing signals associated with the second all-days meta cluster can be assigned to a second person based, at least in part, on one or more first respective distances between a second in-bed center location of the second all-days meta cluster and each predetermined side of the bed area.

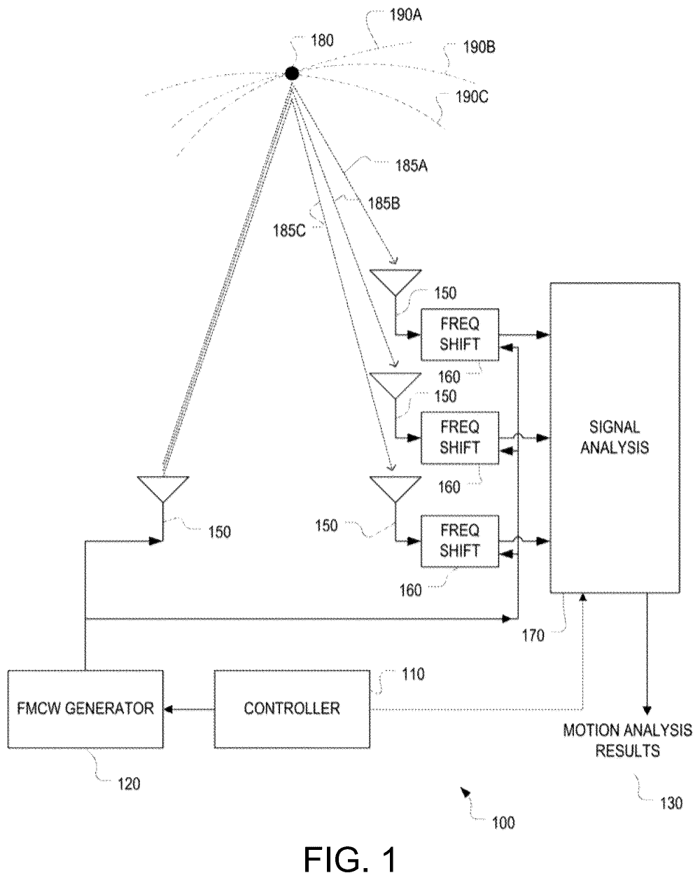

FIG. 1 is a block diagram of a motion tracking system 100 according to an embodiment. The motion tracking system 100 includes a number of antennas 150 that transmit and receive radio frequency signals that are reflected, partially or fully, from objects (e.g., people) in the environment of the system 100, which may include one or more rooms of a building, the interior of a vehicle, etc., and may be partitioned, for example, by substantially radio-transparent barriers, for instance building walls or cloth sheets. In general, the objects in the environment include both fixed objects, such as chairs, walls, etc., as well as moving objects, such as but not limited to people. The system 100 can track people, who may be moving around a room, getting out of a sleep platform (e.g. bed), or may be relatively stationary, for instance, sitting in a chair or lying in bed, but may nevertheless exhibit breathing motion that may be detected by one or more embodiments described below. The system 100 provides motion analysis results 130 based on the radio frequency signals. In various embodiments, these results include locations of one or more people in the environment, detected body or limb gestures made by the people in the environment, detected physical activity including detection of falls, and/or the detected respiration of the people in the environment.

Generally, the system 100 makes use of time-of-flight (TOF) (also referred to as “round-trip time”) information derived for various pairs of antennas 150. For schematic illustration in FIG. 1, three paths 185A-C reflecting off a representative point object 180 are shown between a transmitting antenna 150 and three receiving antennas 150, each path generally having a different TOF. Assuming a constant signal propagation speed c (i.e., the speed of light), the TOF from an antenna at coordinates (xₜ, yₜ, zₜ) reflecting from an object at coordinates (xₒ, yₒ, zₒ) and received at an antenna at coordinates (xᵣ, yᵣ, zᵣ) can be expressed as:

$$\frac{1}{c} ( \sqrt{{( {x}_{t} - {x}_{o} )}^{2} + {( {y}_{t} - {y}_{o} )}^{2} + {( {z}_{t} - {z}_{o} )}^{2}} + \sqrt{{( {x}_{r} - {x}_{o} )}^{2} + {( {y}_{r} - {y}_{o} )}^{2} + {( {z}_{r} - {z}_{o} )}^{2}} ) .$$

For a particular path, the TOF, for example associated with path 185A, constrains the location of the object 180 to lie on an ellipsoid defined by the three-dimensional coordinates of the transmitting and receiving antennas of the path, and the path distance determined from the TOF. For illustration, a portion of the ellipsoid is depicted as the elliptical line 190A. Similarly, the ellipsoids associated with paths 185B-C are depicted as the lines 190B-C. The object 180 lies at the intersection of the three ellipsoids.

Continuing to refer to FIG. 1, the system 100 includes a signal generator that generates repetitions of a signal pattern that is emitted from the transmitting antenna 150. In this embodiment, the signal generator is an ultra-wide band frequency modulated carrier wave (FMCW) generator 120. It should be understood that in other embodiments other signal patterns and bandwidth(s) than those described herein may be used while following other aspects of the described embodiments. The FMCW signals can function as a type of radar that can track relative movement of objections that reflect the FMCW signals. The FMCW generator 120 can receive control signals from a controller 110.

Additional details regarding the motion tracking systems, methods, and/or other aspects of this disclosure are described in the following documents, which are hereby incorporated by reference: U.S. Pat. No. 10,746,852, titled “Vital Signs Monitoring Via Radio Reflections,” which issued on Oct. 18, 2020; U.S. Pat. No. 9,753,131, titled “Motion Tracking Via Body Radio Reflections,” which issued on Sep. 5, 2017; U.S. Patent Application Publication No. 2020/0341115, titled “Subject Identification in Behavioral Sensing Systems,” which published on Oct. 29, 2020; U.S. Patent Application Publication No. 2019/0188533, titled “Pose Estimation,” which published on Jun. 20, 2019; U.S. Patent Application Publication No. 2018/0271435, titled “Learning Sleep Stages From Radio Signals,” which published on Sep. 27, 2018; and/or U.S. Patent Application Publication No. 2017/0311901, titled “Extraction Of Features From Physiological Signals,” which published on Nov. 2, 2017.

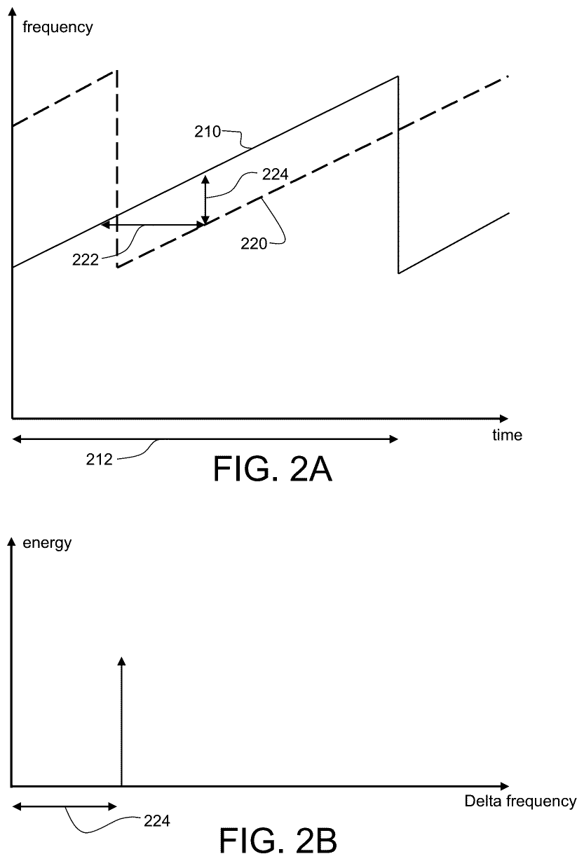

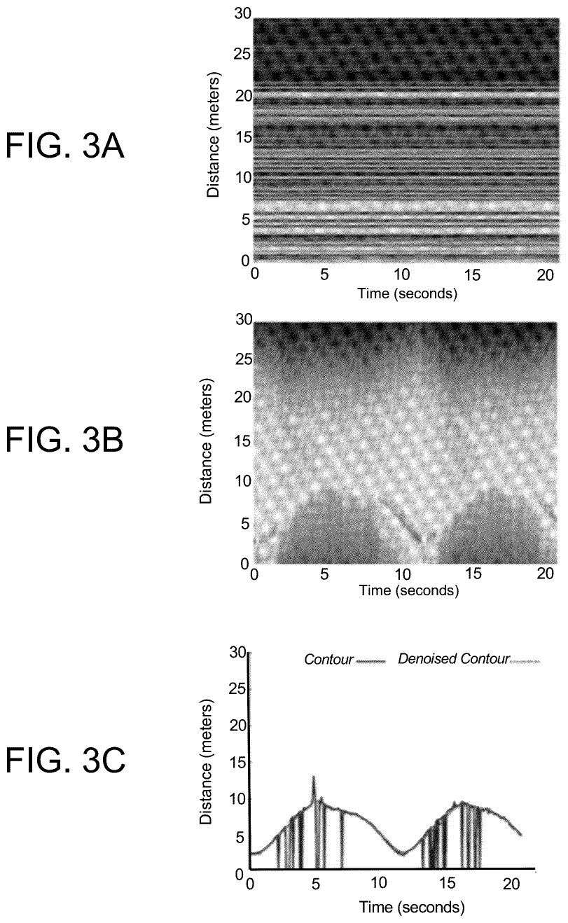

Referring to FIG. 2A, TOF estimates are made using a FMCW approach. Considering a single transmit and receive antenna pair, in each of a series of repeating time intervals 212 of duration T, a transmit frequency is swept from over a frequency range as shown by solid line 210. In some embodiments, the frequency range is about 5.46-7.25 GHZ (i.e., a frequency range of about 1.8 GHZ) with a sweep duration and repetition rate of about 2.5 milliseconds. The receiving antenna receives the signal after a TOF 222 (i.e., reflected from a single object), with frequency as shown in the dashed line 220. Note that the TOF 222 corresponds to a difference 224 in transmitted and received frequencies, which is a product of the TOF and the rate of frequency change of the swept carrier for the transmit antenna.

Referring to FIG. 2B, if the received reflected signal (dashed line 220 in FIG. 2A) is frequency-shifted according to the transmitted signal (solid line 210 in FIG. 2A), then the result will have energy concentrated at the frequency difference 224 corresponding to the TOF. Note that we are ignoring the edges of the intervals 212, which are exaggerated in the figures. Referring back to FIG. 1, a frequency shift component 160 (also referred to as a “downconverter” or a “mixer”) implements the frequency shifting, for example, including a modulator that modulates the received signal with the transmitted signal to retain a low frequency range representing TOF durations that are consistent with the physical dimensions of the environment.

The output of the frequency shifter 160 is subject to a spectral analysis (e.g., signal analysis 170) (e.g., a Fourier transform) to separate the frequency components each associated with a different TOF. In this embodiment, the output of the frequency shifter is sampled and a discrete time Fourier transform implemented as a fast Fourier transform (FFT) is computed for each interval 212. Each complex value of the FFT provides a frequency component with a frequency resolution Δf=n/T_{sweep} where T_{sweep} is the sweep duration (e.g., 2.5 milliseconds) and n is the number of samples per sweep.

Continuing to refer to FIG. 2B, it should be recognized that the distribution of energy over frequency (and equivalently over TOF), is not generally concentrated as shown in this figure. Rather, there is a distribution of energy resulting from the superposition of reflections from the reflective objects in the environment. Some reflections are direct, with the path being direct between the reflecting object and the transmitting and receiving antennas. Other reflections exhibit multipath effects in which there are multiple paths from a transmitting antenna to a receiving antenna via a particular reflecting object. Some multipath effects are due to the transmitted signal being reflected off wall, furniture, and other static objects in the environment. Other multipath effects involve reflection from a moving body, where the path is not direct from the transmitting antenna to a moving object and back to the receiving antenna, but rather is reflected from one or more static objects either on the path from the transmitting antenna to the moving object or from the moving object back to the receiving antenna, or both.

The system 100 addresses the first multipath effect, referred to as static multipath, using a time-differencing approach to distinguish a moving object's reflections from reflections off static objects in the environment, like furniture and walls. Typically, reflections from walls and furniture are much stronger than reflections from a human, especially if the human is behind a wall. Unless these reflections are removed, they would mask the signal coming from the human and prevent sensing her motion. This behavior is called the “Flash Effect”.

To remove reflections from all of these static objects (e.g., walls, furniture), we leverage the fact that since these reflectors are static, their distance to the antenna array does not change over time, and therefore their induced frequency shift stays constant over time. We take the FFT of the received signal every sweep window and eliminate the power from these static reflectors by subtracting the (complex) output of the FFT in a given sweep from the FFT of the signal in a previous sweep. This process is called background subtraction because it eliminates all the static reflectors in the background. In some embodiments, the immediately-previous sweep (i.e., 2.5 milliseconds previous) is background subtracted, while in other embodiments, a greater delay may be used (e.g., 12.5 milliseconds, or even over a second, such as 2.5 seconds) to perform background subtraction.

By eliminating all reflections from static objects, the system is ideally left only with reflections from moving objects. However, as discussed above, these reflections include both signals that travel directly from the transmitting antenna to the moving body (without bouncing off a static object), reflect of the object, and then travel directly back to the receiving antenna, as well as indirect paths that involve reflection from a static object as well as form a moving object. We refer to these indirect reflections as dynamic multi-path. It is quite possible that moving object reflection that arrives along an indirect path, bouncing off a side wall, is stronger than her direct reflection (which could be severely attenuated after traversing a wall) because the former might be able to avoid occlusion.

The general approach to eliminating dynamic multi-path is based on the observation that, at any point in time, the direct signal paths to and from the moving object have travelled a shorter path than indirect reflections. Because distance is directly related to TOF, and hence to frequency, this means that the direct signal reflected from the moving object would result in the smallest frequency shift among all strong reflectors after background subtraction. We can track the reflection that traveled the shortest path by tracing the lowest frequency (i.e., shorted time of flight) contour of all strong reflectors.

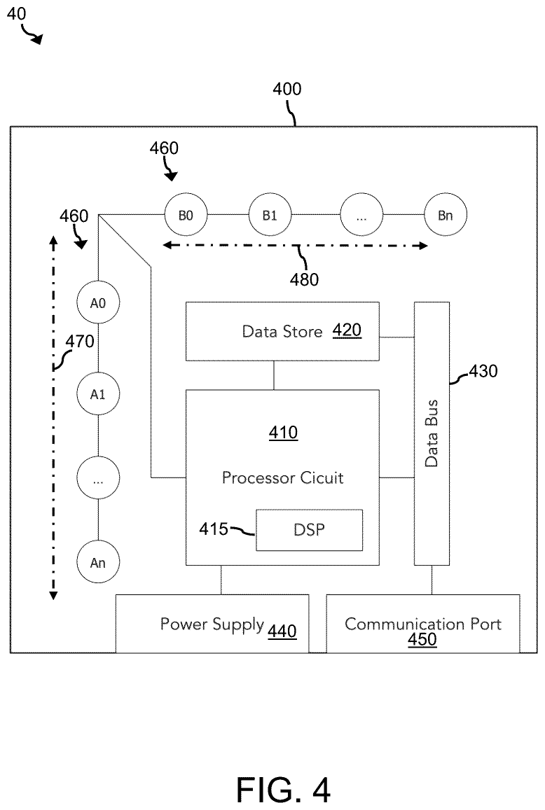

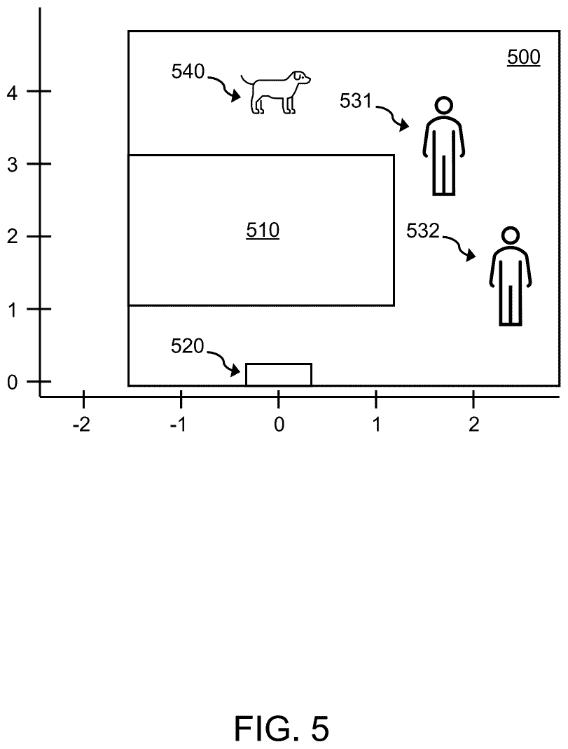

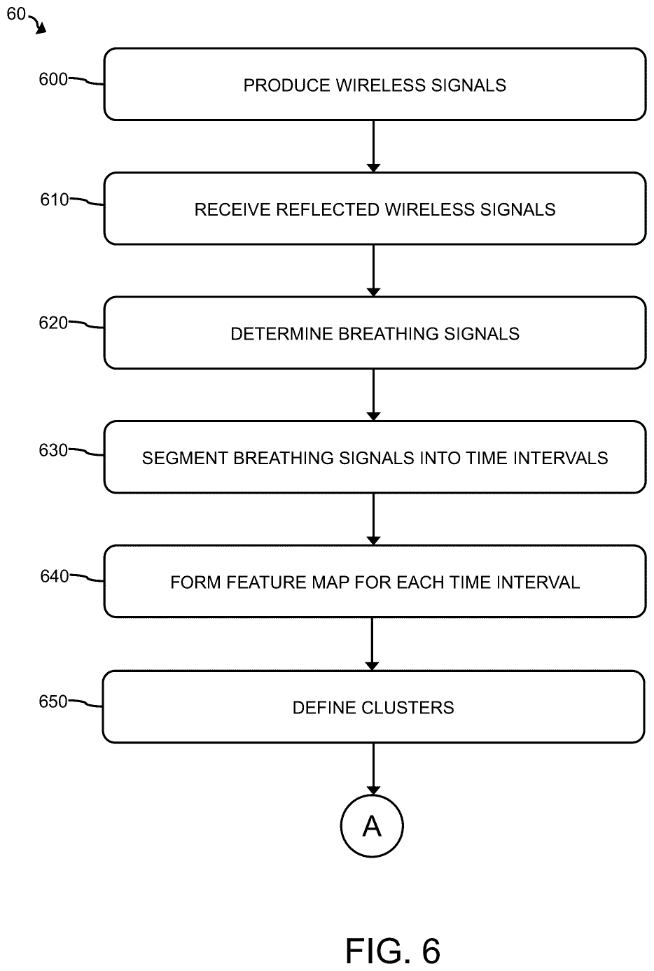

Referring to FIGS. 3A-C, the horizontal axis of each figure represents a time interval of approximately 20 seconds, and the vertical axis represents a frequency range corresponding to zero distance/delay at the bottom to a frequency corresponding to a range of approximately 30 meters at the top. FIGS. 3A and 3B show FFT power before background subtraction (FIG. 3A) and after background subtractions (FIG. 3B).

FIG. 3C shows the successive estimates of the shortest distance (or equivalently time) of flight for successive sweeps, as well as a “denoised” (e.g., smoothed, outlier eliminated, etc.) contour.

To determine the first local maximum that is caused by a moving body, we must be able to distinguish it from a local maximum due to a noise peak. We achieve this distinguishability by averaging the spectrogram across multiple sweeps. In this embodiment, we average over five consecutive sweeps, which together span a duration of 12.5 milliseconds, prior to locating the first local maximum of the FFT power. For all practical purposes, a human can be considered as static over this time duration; therefore, the spectrogram (i.e., spectral distribution over time) would be consistent over this duration. Averaging allows us to boost the power of a reflection from a moving body while diluting the peaks that are due to noise. This is because the human reflections are consistent and hence add up coherently, whereas the noise is random and hence adds up incoherently. After averaging, we can determine the first local maximum that is substantially above the noise floor and declare it as the direct path to the moving body (e.g., a moving human).

In practice, this approach using the first reflection time rather than the strongest reflection proves to be more robust, because, unlike the contour which tracks the closest path between a moving body and the antennas, the point of maximum reflection may abruptly shift due to different indirect paths in the environment or even randomness in the movement of different parts of a human body as a person performs different activities.

Note that the process of tracking the contour of the shortest time of flight is carried out for each of the transmitting and receiving antenna pairs, in this embodiment, for the three pairs each between the common transmitting antenna and the three separate receiving antennas. After obtaining the contour of the shortest round-trip time for each receive antenna, the system leverages common knowledge about human motion to mitigate the effect of noise and improve its tracking accuracy. The techniques used include outlier rejection, interpolation, and/or filtering.

In outlier rejection, the system rejects impractical jumps in distance estimates that correspond to unnatural human motion over a very short period of time. For example, in FIG. 3C, the distance from the object repeatedly jumps by more than 5 meters over a span of few milliseconds. Such changes in distance are not possible over such small intervals of time, and hence the system rejects such outliers.

In interpolation, the system uses its tracking history to localize a person when she stops moving. In particular, if a person walks around a room and then sits on a chair and remains static, the background-subtracted signal would not register any strong reflector. In such scenarios, we assume that the person is still in the same position and interpolate the latest location estimate throughout the period during which we do not observe any motion, enabling us to track the location of a subject even after she stops moving.

In filtering, because human motion is continuous, the variation in an object's distance to each receive antenna should stay smooth over time. Thus, the system uses a filter, such as a Kalman filter, to smooth the distance estimates.

After contour tracking and de-noising of the estimate, the system obtains a clean estimate of the distance travelled by the signal from the transmit antenna to the moving object, and back to one of the receive antennas (i.e., the round-trip distance). In this embodiment that uses one transmitting antenna and three receiving antenna, at any time, there are three such round-trip distances that correspond to the three receive antennas. The system uses these three estimates to identify the three-dimensional position of the moving object, for each time instance.

The system leverages its knowledge of the placement of the antennas. In this embodiment, the antennas are placed in a “T” shape, where the transmitting antenna is placed at the cross-point of the “T” and the receiving antennas are placed at the edges, with a distance of about 1 meter between the transmitting antenna and each of the receiving antennas. For reference, the z axis refers to the vertical axis, the x axis is along the horizontal, and with the “T” shaped antenna array mounted to a wall the y axis extends into the room. Localization in three dimensions uses the intersection of the three ellipsoids, each defined by the known locations of the transmitting antenna and one of the receiving antennas, and the round-trip distance.

Note that in alternative embodiments, only two receiving antennas may be used, for example with all the antennas placed along a horizontal line. In such an embodiment, a two-dimensional location may be determined using an intersection of ellipses rather than an intersection of ellipsoids. In other alternative embodiments, more than three receiving antennas (i.e., more than three transmitting-receiving antenna pairs) may be used. Although more than three ellipsoids do not necessarily intersect at a point, various approaches may be used to combine the ellipsoids, for example, based on a point that is closest to all of them.

For example, the antennas can include an antenna array in which antenna elements are distributed in two dimensions. A particularly useful embodiment is an antenna array in which antenna elements are arranged at regular intervals along vertical and horizontal axes. This arrangement can provide a particularly convenient way to separate signals that come from different locations in the three-dimensional space.

A frame generator can process the data that arrives from the receiving antennas to form RF frames or “frames.” The frame generator can use the antenna elements along the horizontal axis to generate successive two-dimensional horizontal frames and can use the antenna elements along the vertical axis to generate successive two-dimensional vertical frames. The horizontal frames are defined by a distance axis and a horizontal-axis. The vertical frames are defined by the same distance axis and a vertical-angle axis. The horizontal and vertical frames can therefore be viewed as projections of the frame into two subspaces.

The frame generator can generate data indicative of a reflection from a particular distance and angle. It does so by evaluating a double-summation for the horizontal frames and another double-summation for the vertical frames.

The double-summations are identical in form and differ only in details related to the structures of the horizontal and vertical lines of antennal elements and in whether the vertical or horizontal angle is being used. Thus, it is sufficient to show only one of the double summations below:

$$P ( d , θ ) = {\sum }_{n = 1}^{N} {\sum }_{t = 1}^{T} {s}_{n , t} {e}^{j 2 π kdt / c} {e}^{j 2 π nlcos ( θ ) / λ}$$

In the foregoing double summation, P(d, θ) represents the value of the reflection from a distance in the direction θ, s_{n,t} represents the t^{th} sample of the reflected chirp (e.g., transmitted signal pattern) as detected by the nth antenna element in the line of antenna elements, c and λ represent the radio wave's velocity and its relevant wavelength, respectively, N represents the total number of antenna elements in the relevant axis of antenna elements, T represents the number of samples from the relevant reflection of the outgoing chirp, l represents the spatial separation between adjacent antenna elements, and k represents the slope of the chirp in the frequency domain.

As a result, at each step, it is possible to represent the reflected signal using its projection on a horizontal plane and on a vertical plane. The horizontal frame captures information concerning a subject's location and the vertical frame captures information concerning the subject's build, including such features as the subject's height and girth. Differences between successive frames in a sequence of horizontal and vertical frames can provide information concerning each subject's characteristic gait and manner of movement. A preferred embodiment operates at a frame rate of thirty frames-per-second. This is sufficient to assume continuity of locations. Additional details regarding the antenna arrangement and frame calculation are disclosed in U.S. Patent Application Publication No. 2020/0341115.

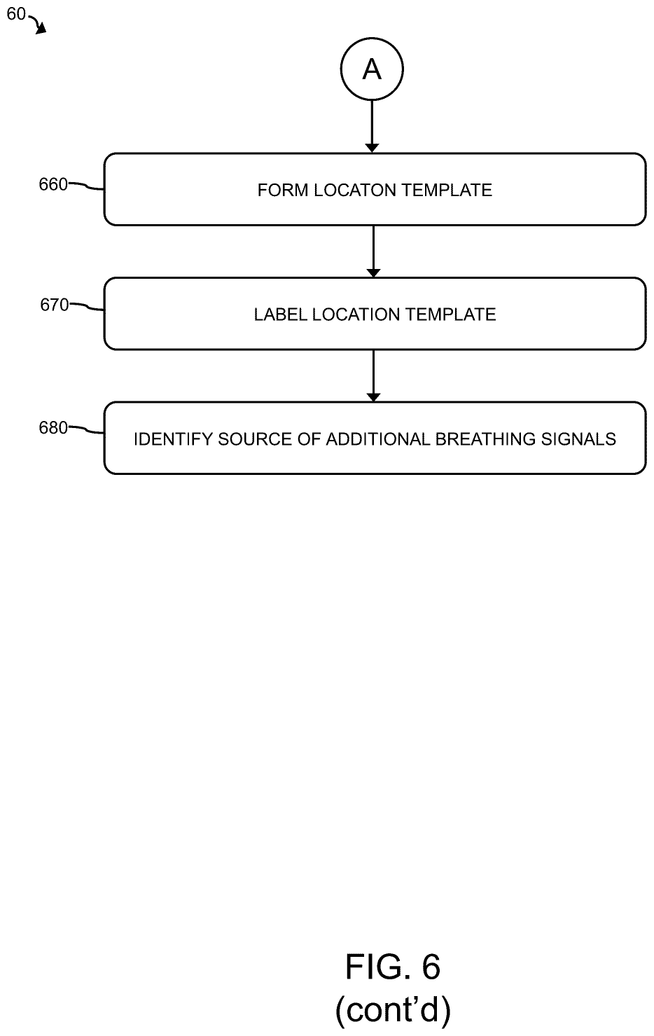

FIG. 4 is a block diagram of a motion tracking system 40 according to another embodiment. The motion tracking system 40 can be the same as motion tracking system 100. The motion tracking system 40 includes a housing 400, a processor circuit 410, a data store 420, a data bus 430, a power supply 440, an optional communication port 450, and a plurality of RF antennas 460. The housing 400 can contain some or all components of the system 40 including the processor circuit 410, the data store 420, the data bus 430, the power supply 440, the optional communication port 450, and/or the RF antennas 460. The housing 400 can comprise plastic, ceramic, or another material. The housing 400 is preferably at least partially transparent to the RF frequency(ies) emitted and/or received by the RF antennas 460. For example, the housing 400 can provide minimal (e.g., less than 5-10%) or no attenuation of the RF signals emitted and/or received by the RF antennas 460. In some embodiments, some or all of the components can be mounted on or electrically connected to a printed circuit board. In some embodiments, the RF antennas 460 can be replaced another energy-emission device such as ultrasonic transducers.

The processor circuit 410 can comprise an integrated circuit (IC) such as a microprocessor, an application-specific IC (ASIC), or another hardware-based processor. For example, the microprocessor can comprise a central processing unit (CPU), a graphics processing unit (GPU), and/or another processor. The processor circuit 410 also includes one or more digital signal processors (DSPs) 415 that can drive the RF antennas 460. The construction and arrangement of the processor circuit 410 can be determined by the specific application and availability or practical needs of a given design. The processor circuit 410 is electrically coupled to the power supply 440 to receive power at a predetermined voltage and form. For example, the power supply 440 can provide AC power, such as from household AC power, or DC power such as from a battery. The power supply 440 can also include an inverter or a rectifier to convert the power form as necessary (e.g., from AC power to DC power or from DC power to AC power, respectively).

The optional communication port 450 can be used to send and/or receive information or data to and/or from a second device, such as a computer, a sensor (e.g., in the subject's bedroom), a server, and/or another device. The optional communication port 450 can provide a wired or wireless connection for communication with the second device. The wireless connection can comprise a LAN, WAN, WiFi, cellular, Bluetooth, or other wireless connection. In some embodiments, the optional communication port 450 can be used to communicate with multiple devices.

The data store 420 can include computer-readable memory (e.g., volatile and/or non-volatile memory) that can store data representing stored machine-readable instructions and/or data collected by the system 40 or intermediate data used by the processor circuit 410. The data bus 430 can provide a data connection between the data store 420, the processor circuit 410, and/or the optional communication port 450. The data store 420 can also include program instructions that represent artificial intelligence, a trained neural network (e.g., a convolutional neural network, a deep neural network), and/or machine learning that can be trained to perform one or more tasks of the technology.

The RF antennas 460 can include or consist of one or more dedicated transmitting antennas and/or one or more dedicated receiving antennas. Additionally or alternatively, one or more of the RF antennas 460 can be a transceiver antenna (e.g., that can transmit and receive signals but preferably not simultaneously). Any receiving antenna 460 can receive a signal from any transmitting antenna 460. In one example, an antenna sweep can occur by sending a first signal from a first transmitting antenna 460, which is received by one or more receiving antenna(s) 460. Next, a second transmitting antenna 460 can send a second signal, which can be received by one or more receiving antenna(s) 460. This sweep process can continue where each transmitting antenna 460 sequentially sends a respective signal that is received by one or more receiving antenna(s) 460. Each signal can be reflected by the subject partially or fully.

The RF antennas 460 can be disposed in a suitable arrangement with respect to one another to form one or more wireless transmit-receive arrays. As illustrated, a first group or array of RF antennas 460 (A0, A1 . . . . An) is placed along or parallel to a first axis 470 and a second group or array of RF antennas 460 (B0, B1 . . . . Bn) is placed along or parallel to a second axis 480 that is orthogonal to the first axis 470, for example in an “L” arrangement. The first and second groups of RF antennas 460 can be configured and arranged at to capture a spatial position of the target of interest, e.g., the subject's body and the bed. It is noted that the first and second groups of RF antennas 460 can be arranged in different relative orientations to achieve the same or substantially the same result. For example, the first and second axes 470, 480 can be oriented at other angles with respect to each other that are different than 90°. Alternatively, the first and second groups of RF antennas 460 can be configured in a “T” arrangement or a “+” instead of the “L” arrangement illustrated in FIG. 4.

In addition or in the alternative, one group of RF antennas 460 can be used to collect reflection and position data in the (x, y) plane, which can be defined as parallel to the floor or sleeping surface planes, and the other group of RF antennas 460 can be used to optionally collect height or elevation data regarding the third (2) axis position. In another embodiment, each group of RF antennas 460 can include one or more dedicated transmit antennas, one or more dedicated receive antennas, and/or one or more dedicated transmit antennas and one or more dedicated receive antennas. The RF antennas 460 in each group can be spaced at equal distances from each other, or they may not be, and may even be randomly spaced or distributed in the housing 400. Alternatively, those skilled in the art will understand that the system may be constructed using more than one physical device that can be distributed spatially about an indoor environment such as a bedroom, hospital room or other space, i.e., placing separate components within multiple distinct housings.

In a preferred but not limiting example, the RF antennas 460 can span a finite spatial extent which allows for localization or triangulation to determine the position of a reflecting body, e.g., the subject's body. For example, the RF antennas 460 can emit RF signals with a wavelength in a range of about 0.01 meter (i.e., about 1 cm) to about 1 meter, including any value or range therebetween. In another example, the RF antennas 460 can emit RF signals with a wavelength in a range of about 1 cm to about 10 cm, including any value or range therebetween. Electromagnetic radiation waves with wavelengths greater than 1 mm are preferred, i.e., those wavelengths substantially greater than the wavelengths of visible light.

The motion tracking system 40 can be programmed, configured, and/or arranged to perform motion tracking automatically, for example using the RF antennas 460 and processing components. Therefore, the motion tracking system 40 can continuously and inexpensively monitor a human subject in an indoor environment such as a bedroom, though sometimes the present system can emit and receive wireless signals through walls, furniture or other obstructions to extend its versatility and range. Also, the motion tracking system 40 can continuously monitor, detect, and/or and measure the data required to determine and track the respiration of one or more subjects under observation and/or to distinguish the respiration of multiple subjects under observation. Furthermore, the motion tracking system 40 can be deployed to an observation space (e.g., a subject's bedroom) in a non-obtrusive form factor such as a bedside apparatus or an apparatus inconspicuously mounted on a wall, ceiling, or fixture of the subject's room.

The motion tracking system 40 can be coupled to a communication network (e.g., using optional communication port 450) so that data collected may be analyzed remotely, off-line, or in real time by a human or a machine (e.g., computer, server, etc.) coupled to the network. For example, the optional communication port 450 can be used to deliver data over the network (e.g., the internet) to a server, clinical station, medical care provider, family member, or other interested party. Data collected can be archived locally in memory (e.g., data store 420) of the system 40 or may transmitted (e.g., using optional communication port 450) to a data collection or storage unit or facility over a wired or wireless communication channel or network.

A processor-implemented or processor-assisted method can be carried out automatically or semi-automatically. The processing may take place entirely on internal processing components in the system 40 and/or at a remote processing location. For example, the system 40 can receive the wireless signal data and send that data to be processed remotely such as at a cloud-based server. In yet other embodiments a hybrid arrangement may be used where processing is performed at both the local device (e.g., in system 40) and remotely. Therefore, for the present purposes, unless described otherwise, the location of the processing acts is not material to most or all embodiments.

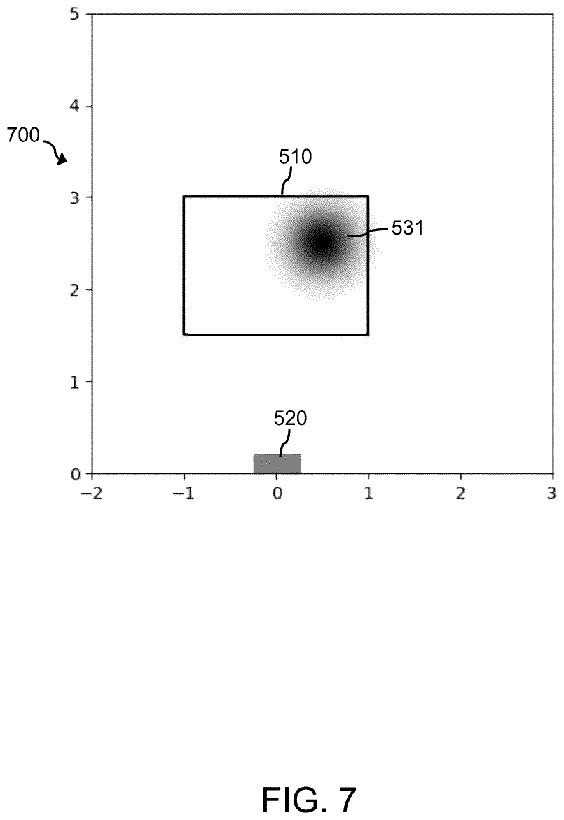

FIG. 5 is a block diagram of an example room 500 or other indoor environment in which embodiments of the technology can be applied. A typical home or institution or hospital room may be the general space within which most or all of the present steps are carried out. The room 500 includes a bed 510 or other sleep platform and a wireless motion tracking system 520 disposed at a (0, 0) reference location, where the numbers along the two axes are indicated in meters. The wireless motion tracking system 520 can be the same as system 100 and/or system 40. The area and position of the walls of the room 500 and its contents, including the bed 510 and/or other furnishings, can be automatically measured by the wireless motion tracking system 520, or can be entered manually, such as at the time of installation of the system 520, to identify or key in the relative dimensions or locations of key objects such as walls, beds, and so on.

Through data collection, observation, and analysis, the system 520 can determine or estimate the breathing (e.g., respiration), physical position, activity state (e.g., asleep, awake), sleep stage, and/or other health indicia of people 531, 532 located in the room 500. The system 520 can also automatically track each person 531, 532 as they move within the room 500 and can distinguish each person 531, 532 from one another, from objects within the room, such as the bed 510 or other furniture, and from pets 540.

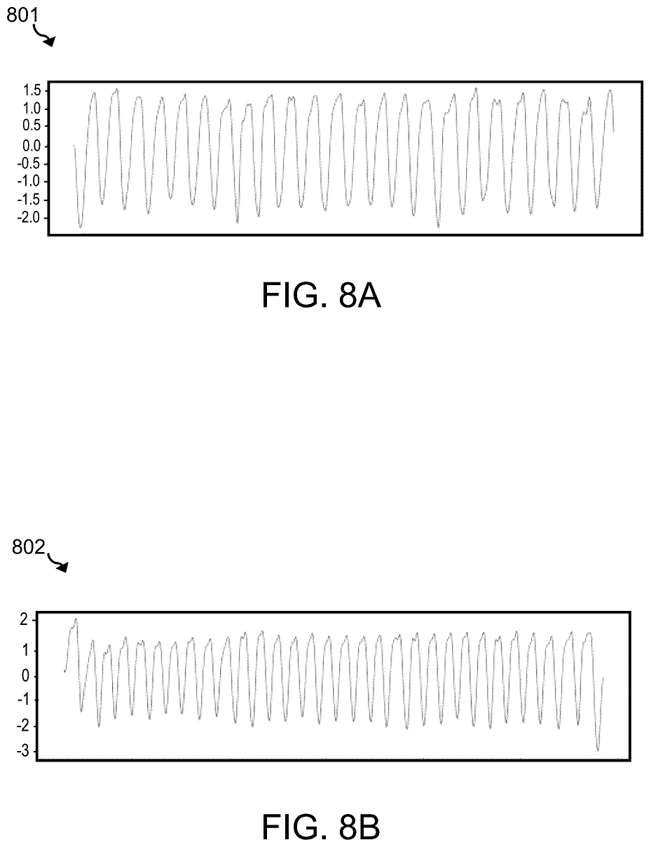

FIG. 6 is a flow chart of a method 60 for automatically determining the source of breathing signals from multiple people according to an embodiment. Method 60 can be implemented using motion tracking system(s) 100, 40, and/or 520.

In step 600, FMCW wireless signals are produced by one or more transmitting antennas. The transmitting antenna(s) can be the same as antennas 150, 460. At least some of the wireless signals are transmitted towards one or more people, such as towards people 531, 532 in room 500.

In step 610, reflected FMCW wireless signals are received by one or more receiving antennas. The reflected wireless signals can be reflected by one or both people 531, 532, walls in the room 500, and/or furniture (e.g., bed 510) in the room 500. The receiving antennas can be the same as antennas 150, 460. The receiving antenna(s) can be the same as or different than the transmitting antenna(s). In addition, the number and/or arrangement of the receiving antenna(s) can be the same as or different than the number and/or arrangement of the transmitting antenna(s). The reflected FMCW wireless signals (e.g., FMCW data) can function as a type of radar.

Initially, the FMCW wireless signals can be produced, received, and collected over a limited period of time (e.g., over several days) to provide calibration data, which can be used as an input to create location templates.

In step 620, breathing signals of one or more people (e.g., person 531 and/or person 532) are determined using the reflected wireless signals.

The FMCW data can include a series of snapshots collectively called a “heatmap.” As an example, a heatmap might consist of a series of snapshots taken at a rate of 5 snapshots per second. In the example scenario, each snapshot comprises a three-dimensional map of the room, discretized into individual voxels. A numerical value, in real or complex form, is associated with each voxel, indicating the strength and phase of the signal reflected from that voxel.

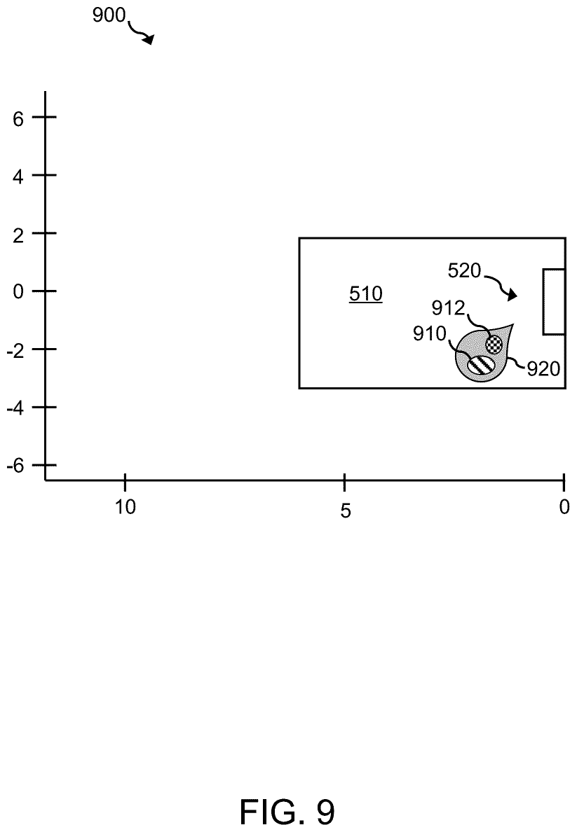

FIG. 7 illustrates an example heatmap 700 of accumulated position data (illustrated as dark color) indicating that a person 531 is located in bed 510 for the majority of the observation time. The wireless motion tracking system 520 can therefore observe and locate the subject(s) during his or her sleep. In other embodiments, the heatmap can indicate that the person 531 is out of bed 510 and moving in the room 500, and/or that another person 532 and/or pet 540 (not illustrated) is/are in the room 500.

Conclusions can be drawn regarding the person's sleep pattern and health from the aggregated position data, for example indicating how much the subject moves around in the sleep or lying-down area during a night's sleep or from night to night, and/or the sleep stage of the person. Conclusions can also be drawn regarding the person's respiration, movement within the room 500, and/or other motions.

A person's breathing signal is a time-series signal reflecting the intake of air into the lungs. As an example, the signal might indicate the volume of air in the lungs at each instant in time. A breathing signal can be extracted from a heatmap by observing changes in the voxel values from snapshot to snapshot. In particular, as a person's chest and abdomen is displaced by the motion inherent in breathing, the magnitude and/or phase of the radio waves reflected from the corresponding voxels changes. For example, if a person is breathing at a rate of 20 breaths per minute, the phase of the radio signal reflecting from the person's chest might oscillate with a period of 3 seconds. This oscillation in phase can be interpreted as the person's breathing signal.

But identifying which breathing signals belong to which people in a room is complicated by several factors. First, radio waves can bounce off objects and walls, and hence a person's breathing signal may appear to originate at one or more voxels in the heatmap other than the voxel or voxels where the person's chest is located. Second, radio waves add constructively, so that the signal reflected by an individual voxel may be a combination of the breathing signals of two people. Third, pets and other moving objects such as fans may also produce periodic changes in voxel magnitude and phase that mimic human breathing.

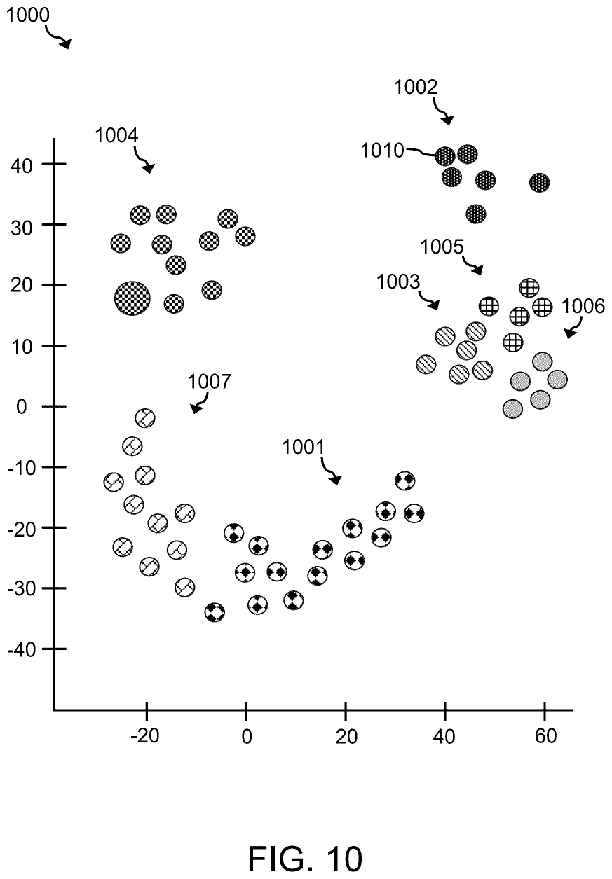

Signals that have been added constructively can be separated using techniques such as independent component analysis (ICA). ICA operates on a set of inputs (e.g., the time series data from each of the voxels in a heatmap), each of which may be a linear combination of independent underlying signals. ICA can extract one or more breathing signals from the time series data. An example of two breathing signals 801, 802 extracted from a heatmap using ICA are illustrated in FIGS. 8A and 8B, respectively. Given two or more such signals 801, 802, location templates can aid in identifying which signals belong to which people.

In step 630, the breathing signal data are segmented into predetermined time intervals, such as 3-minute to 5-minute time intervals. In an embodiment, the breathing signal data are segmented into 4-minute time intervals.

In step 640, a feature map is created for each time interval. In an embodiment, a set of “candidate” breathing signals is extracted from the heatmap data for each time interval. The set of candidate breathing signals can be extracted using ICA or another technique. For each time interval, each candidate breathing signal is then correlated with each voxel. For example, a Pearson correlation coefficient may first be computed between a respective candidate breathing signal and the heatmap data for each voxel, and then the absolute values of the Pearson correlation coefficients may be taken so that all correlation scores are non-negative. For each candidate breathing signal, the correlation score at each voxel may also be weighted by some measure of the power of the reflections from the voxel, e.g., by multiplying the correlation score by the average of the square of the magnitude of the voxel value over the course of the interval. The result of this calculation for each voxel is called a feature map. For each interval, there is a feature map for each candidate breathing signal. The feature map provides a numerical value, e.g., a non-negative real number, for each voxel.

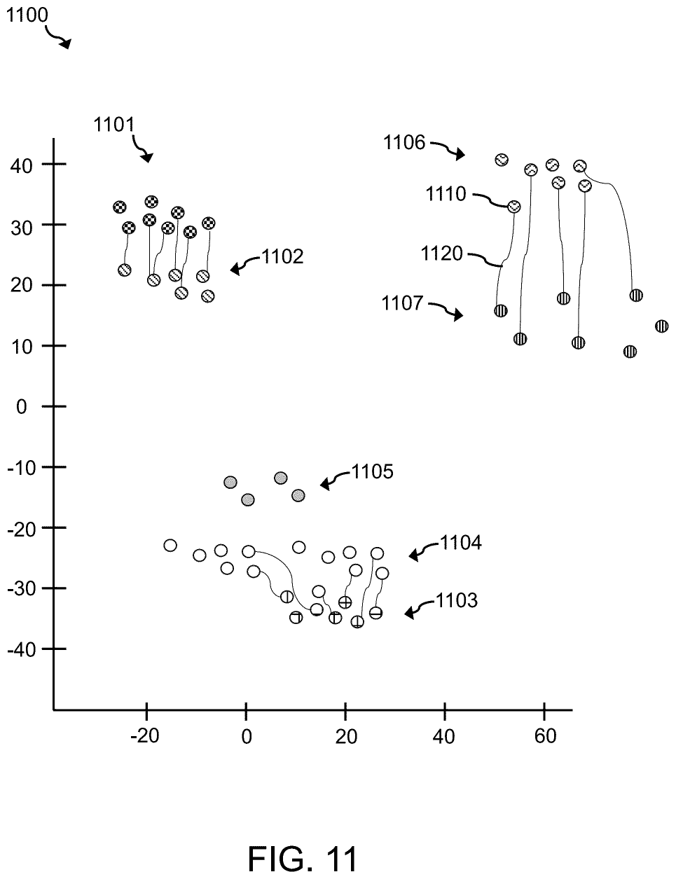

FIG. 9 illustrates an example feature map 900 for one candidate for one interval according to an embodiment. For purposes of illustration, the heatmap data, and hence the feature map, is two-dimensional rather than three-dimensional. Thus, the example feature map 900 is partitioned in two dimensions into pixels rather than in three dimensions into voxels. Higher scores are represented as patterns 910, 912, while lower scores are represented as solid gray 920. In practice, the heatmap data and the feature map can be partitioned in three dimensions as voxels. The wireless motion tracking system 520 is located midway on the rightmost edge of the diagram, facing bed 510. The area where the heatmap data shows the highest correlation with the candidate breathing signal is in the bed 510 and is represented as pattern 910. The bed 510 is overlaid on the feature map 900 but may not be included in some embodiments.

For any given interval, the feature maps for different candidate breathing signals are typically distinct. For example, if a first candidate breathing signal is for a person sleeping on the left side of a bed, the feature map for that candidate is expected to show higher scores for the voxels on the left side of the bed, as well as possibly at other locations around the room, depending on the reflections of the radio waves. Similarly, the feature map for a second candidate breathing signal corresponding to a second person, sleeping on the right side of the bed, might show higher scores for voxels on the right side of the bed. But the second person might be occluded from direct line of sight by the first person, in which case the feature map for the candidate corresponding to the second person might show correlations only with voxels at some distance from the bed.

In step 650, clusters are defined for the feature maps. Clusters can be used to group the candidate breathing signals in the feature maps for all candidates for some or all time intervals.

An optional first step in defining clusters is dimensionality reduction. The values of the individual voxels in the feature map, along with the average breathing rate of the corresponding candidate, are viewed as the input variables. A dimensionality reduction technique such as principal component analysis (PCA) can be used to project these input variables onto a smaller dimensional space, i.e., to map these input variables to a smaller number of output variables.

Regardless of whether dimensionality reduction has been performed, the next step in the clustering process is to define a function that specifies the “distance” between any pair of feature maps. For example, the distance between two feature maps might be computed as the L2 norm, which is also known as the Euclidean-norm or Euclidean distance. Given this distance function, another dimensionality reduction algorithm such as t-distributed stochastic neighbor embedding (t-SNE) is used to map the feature maps to two dimensions.

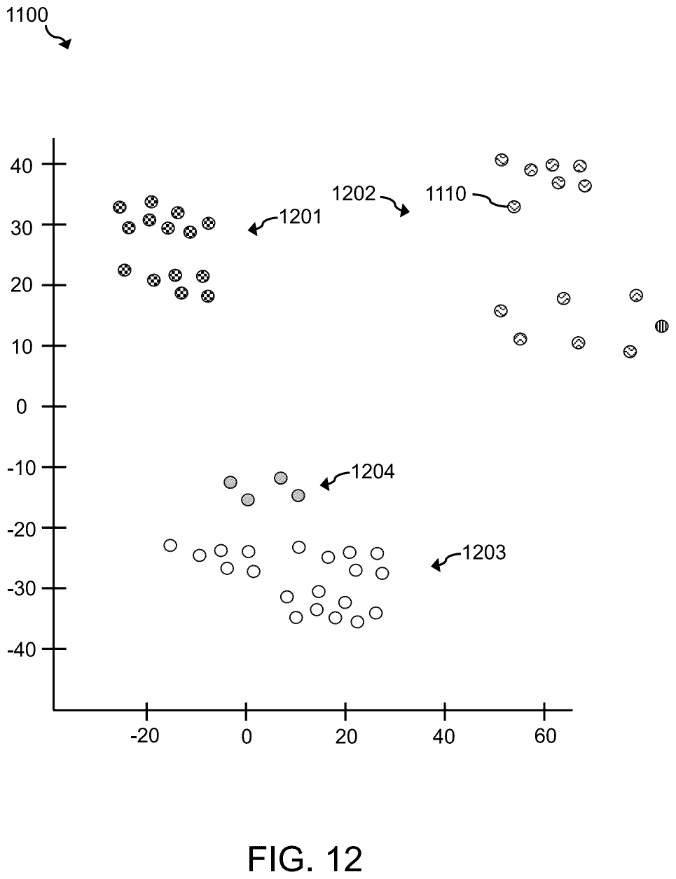

Once the feature maps have been mapped to two dimensions, they are formed into clusters, with the intent that, within any one cluster, all of the feature maps (e.g., breathing signals) belong to the same person. At this stage several distinct clusters may belong to the same person. If so, these clusters can optionally be merged later. Feature maps may be clustered using a technique such as k-means clustering, again using the L2 norm as a distance function. An example of a clustered feature map 1000 is illustrated in FIG. 10, where the number of clusters, k, has been chosen to be 7. Each cluster 1001-1007 is illustrated with feature maps 1010 of each cluster having a different pattern or color.

Another automated approach to clustering is to view each feature map as a node in a graph, with two nodes connected by an edge whose length is determined by the distance function. Optionally, an edge may be excluded from the graph if the distance is too large. Graph partitioning techniques can then be used to divide the graph into clusters.

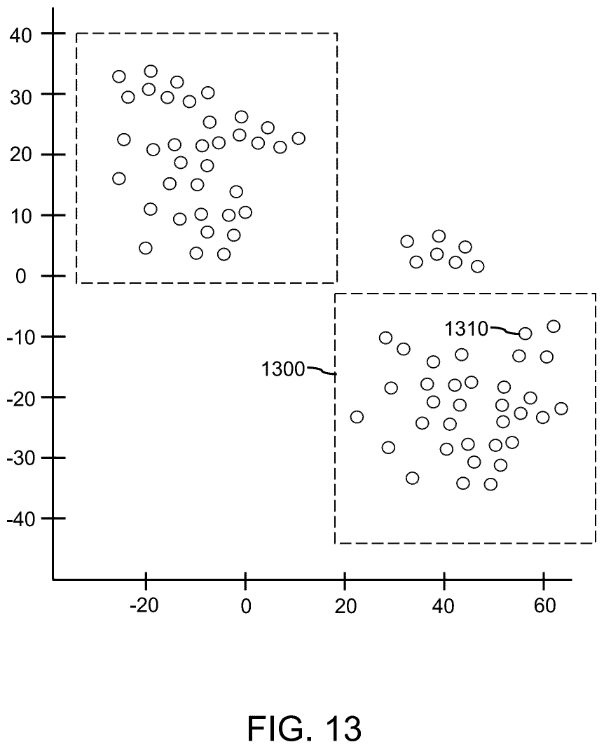

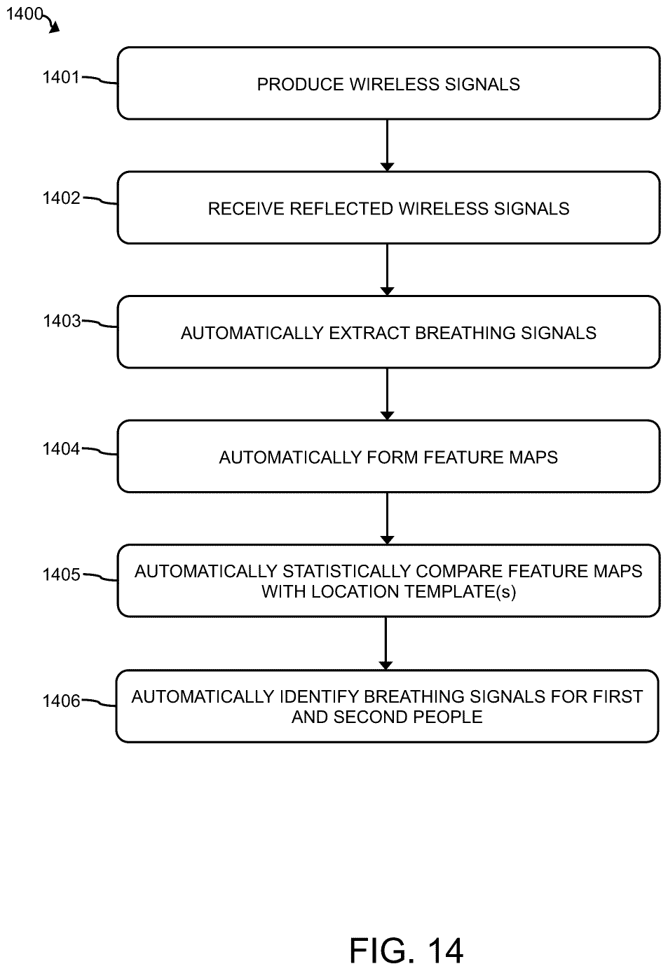

A hybrid clustering approach is also possible. For example, the feature maps can be partitioned into clusters using the k-means algorithm, and then two clusters can be merged if they are connected by enough edges. FIG. 11 illustrates a clustered feature map 1100 where the feature maps 1110 are initially partitioned into clusters 1101-1107 with the feature maps 1110 in each cluster have different pattern or color. In this example, k has been chosen as 7. The clusters with feature maps 1110 connected by enough edges (shown as example lines 1120) are merged. The final set of 4 clusters 1201-1204, after merger, is illustrated in FIG. 12.

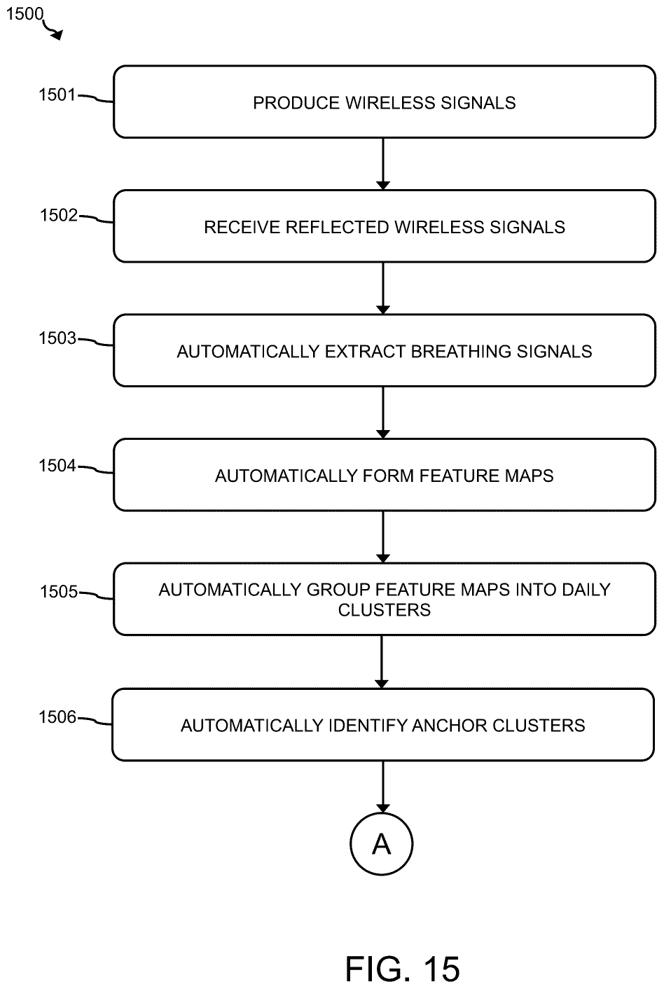

Alternatively, as shown in FIG. 13, clusters can be formed by manual labeling. For example, a person can draw one or more polygons 1300 in the two-dimensional plane to enclose the feature maps 1310 belonging to one cluster.

In a typical application, breathing signal data is collected every day over a period of days. In one example, the feature maps for all days are combined into a single set before forming clusters.

In step 660, a location template is formed.

In one embodiment, after clustering, the feature maps within each cluster are averaged together to form a single average feature map. Such an average feature map looks the same or similar to the feature map illustrated in FIG. 9. Optionally, statistics concerning the corresponding candidate breathing signals are computed as well. For example, the breathing rates of the corresponding candidate breathing signals may be averaged together. In addition, other statistics such as the standard deviation of the breathing rates may be computed. The average feature map and these statistics form a location template.

In another embodiment, the location template does not contain an average feature map. Instead, the location template maintains the clusters of the feature maps of the data that were initially collected.

In step 670, the location template is labeled.

A human can examine the location template (e.g., average feature map or the clusters of the feature maps of the data that were initially collected) and, based on the location of the voxels and their correlations with the candidate breathing signals, as indicated by the colors in FIG. 9, and the optional breathing statistics, the human can label the location template with the identity of the person the feature map represents. For example, if the feature map corresponding to cluster 4 in FIG. 10 shows high correlations with voxels on the near side of the bed, and person 1 sleeps on that side of the bed, then it might be assigned the label “person 1.” Alternatively, the human labeler may decide that the feature map represents something other than a person, e.g., a pet or a fan, and labels it as such. For example, the feature map corresponding to cluster 1 in FIG. 10 shows high correlations corresponding to voxels where a floor fan is located, and if the “breathing rate” is rapid compared to that of a typical person, then the location template might be assigned the label “fan.” Alternatively, the system can automatically label the clusters with breathing rates within the normal human range (e.g., 10-20 breaths per minute) as people, and can label clusters with breathing rates outside the normal range as non-human.

In step 680, the identity of additional breathing signals received by the system are determined using one or more labeled location templates.

The identity to whom a breathing signal belongs can be determined by comparing the additional breathing signals to one or more labeled location templates and selecting the best match.

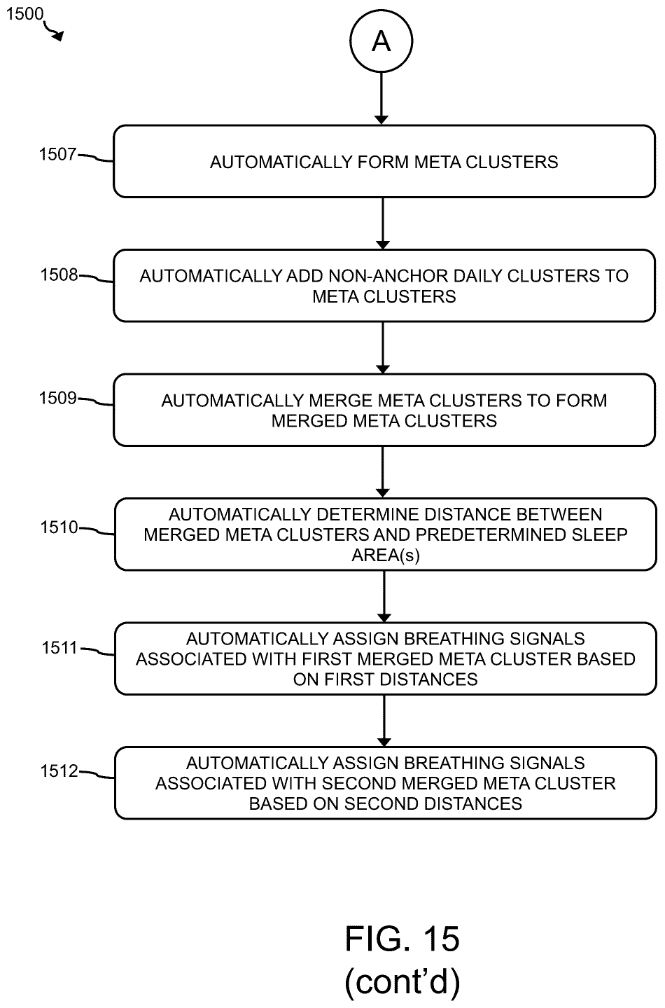

FIG. 14 is a flow chart of a method 1400 for automatically determining the source of breathing signals from multiple people according to an embodiment. Method 1400 can be implemented using motion tracking system(s) 100, 40, and/or 520.

In step 1401, FMCW wireless signals are produced by one or more transmitting antennas over a time period. The transmitting antenna(s) can be the same as antennas 150, 460. At least some of the wireless signals are transmitted towards one or more people, such as towards people 531, 532 in room 500. A computer or a processor (e.g., processor circuit 410) can produce control signals that can cause the transmitting antenna(s) to produce the FMCW wireless signals. Step 1401 can be the same as step 600.

In step 1402, reflected FMCW wireless signals are received by one or more receiving antennas over the time period. The reflected wireless signals can be reflected by one or both people 531, 532, walls in the room 500, furniture (e.g., bed 510) in the room 500, and/or other objects in the room 500. The receiving antennas can be the same as antennas 150, 460. The receiving antenna(s) can be the same as or different than the transmitting antenna(s). In addition, the number and/or arrangement of the receiving antenna(s) can be the same as or different than the number and/or arrangement of the transmitting antenna(s). The reflected FMCW wireless signals (e.g., FMCW data) can function as a type of radar. The reflected FMCW wireless can be received by the computer or a processor (e.g., processor circuit 410). Step 1402 can be the same as step 610.

In step 1403, breathing signals of one or more people (e.g., person 531 and/or person 532) are automatically extracted from the reflected wireless signals (e.g., from the FMCW data). The breathing signals can be automatically extracted by a computer or a processor (e.g., processor circuit 410) in electrical communication with the receiving antenna(s). The breathing signals can be extracted using ICA or another technique. Step 1403 can be the same as step 620.

In step 1404, feature maps are automatically formed for the breathing signals. The feature maps represent a correlation of the breathing signals at each voxel in a plurality of voxels that represent three-dimensional locations within the room 500. Each feature map represents a respective time interval of the time period, where the time period is subdivided into a plurality of time intervals. The time period can be subdivided equally into the time intervals. Alternatively, some of the time intervals can have a different duration than other time intervals. Step 1404 can be the same as step 630.

In step 1405, the feature maps are automatically statistically compared with one or more location templates. The location template(s) can comprise the location template(s) formed in step 660. Additionally or alternatively, the location template(s) can comprise the location template(s) labelled in step 670.

In step 1406, breathing signals for a first person and breathing signals for a second person are automatically identified based, at least in part, on statistical correlations between each location template and each feature map.

In one example, the location template(s) include a first location template and a second location template. The first location template includes a first correlation of the collected breathing signals for a first person at each voxel. The second location template includes a second correlation of the collected-breathing signals for a second person at each voxel. In step 1405, each feature map is automatically statistically compared with the first location template and with the second location template. In step 1406, the breathing signals for each feature map having a higher statistical correlation with the first location template compared to with the second location template can be identified as belonging to (or assigned to) the first person. In addition, the breathing signals for each feature map having a lower statistical correlation with the first location template compared to with the second location template can be identified as belonging to (or assigned to) the second person.

Additionally or alternatively, the statistical comparison in step 1405 can include calculating a respective average breathing rate of the breathing signals associated with each feature map. The respective average breathing rate is then statistically compared with the average breathing rate of the breathing signals associated with each location template, which can be referred to as an average location-template breathing rate. In step 1406, the statistical correlations can include a first statistical comparison or comparison of the respective average breathing rate for each feature map with a first average location-template breathing rate of the first location template. The statistical correlations can also include a second statistical correlation or comparison of the respective average breathing rate for each feature map with a second average location-template breathing rate of the second location template. The first and second correlations can include Pearson correlations. The breathing signals for each feature map having an average breathing rate having a higher statistical correlation with the first average location-template breathing rate compared to with the second average location-template breathing rate can be identified as belonging to (or assigned to), or more likely to belong to, the first person. In addition, the breathing signals for each feature map having an average breathing rate having a lower statistical correlation with the first average location-template breathing rate compared to with the second average location-template breathing rate can be identified as belonging to (or assigned to), or more likely to belong to, the second person.

Additionally or alternatively, some or all of the feature maps can be grouped into clusters, for example in the same or similar manner as in step 650. The breathing signals can then be identified based, at least in part, on statistical correlations between each location cluster and each feature map. The statistical correlations can a statistical correlation of the average breathing rate of each cluster and each average location-template breathing rate.

In another example, the location template(s) includes only one location template (or only one location template is used to identify the source of the breathing signals). In step 1405, each feature map is automatically statistically compared with the location template. In step 1406, the breathing signals for each feature map having a statistical correlation higher than a threshold value can be identified as belonging to (or assigned to) the first person. The breathing signals for each feature map having a statistical correlation lower than or equal the threshold value can be identified as belonging to (or assigned to) the second person.

Additionally or alternatively, the statistical comparison in step 1405 can include calculating a respective average breathing rate of the breathing signals associated with each feature map. The respective average breathing rate is then statistically compared with the average breathing rate of the breathing signals associated with the average location-template breathing rate. In step 1406, the statistical correlations can include a statistical correlation (e.g., a Pearson correlation) or comparison of the respective average breathing rate for each feature map with the average location-template breathing rate of the location template. The breathing signals for each feature map having an average breathing rate having a statistical correlation with the average location-template breathing rate that is higher than a threshold value can be identified as belonging to (or assigned to), or more likely to belong to, the first person. The breathing signals for each feature map having an average breathing rate having a statistical correlation with the average location-template breathing rate that is lower than or equal to the threshold value can be identified as belonging to (or assigned to), or more likely to belong to, the second person.

Additionally or alternatively, some or all of the feature maps can be grouped into clusters, for example in the same or similar manner as in step 650. The breathing signals can then be identified based, at least in part, on statistical correlations between each location cluster and each feature map. The statistical correlations can a statistical correlation of the average breathing rate of each cluster and the average location-template breathing rate.

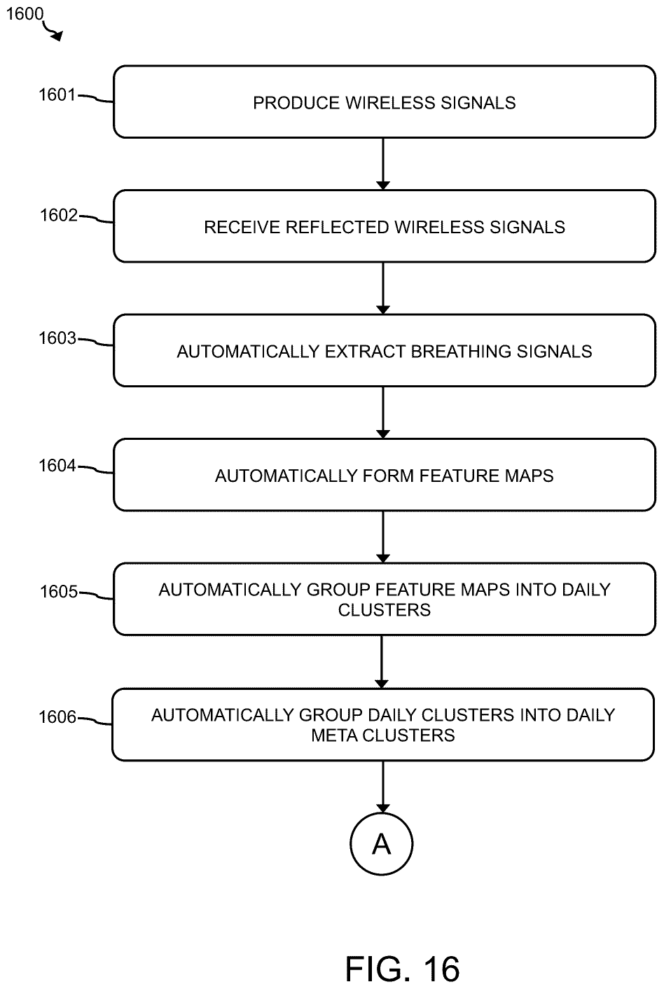

FIG. 15 is a flow chart of a method 1500 for automatically determining the source of breathing signals from multiple people according to another embodiment. Method 1500 can be implemented using motion tracking system(s) 100, 40, and/or 520.

In another example, the method examines a time window spanning multiple days (e.g., 2 days to 30 days or another number of days) and computes feature maps for each of the days in the time window. For each day clusters are formed using the feature maps for that day. The method then merges clusters from the same day and across different days if they are determined to belong to the same person.

In this example, breathing signals are collected in a bedroom where at most two people sleep. These people are referred to as person 1 and person 2. Person 1 is the primary person under observation and is assumed to spend more time in the room. The method assigns each breathing signal to at most one of the two people. The “location template” for this algorithm includes or consists of the location and size of the bed in the room (called the “bed area”) and the respective sides of the bed on which the people sleep. Within the bed, the area on the side of the bed where a person sleeps is called that person's “sleep area.”

For each feature map, an “in-bed feature map” can be calculated by weighting the values of the feature-map pixels in the bed area more heavily than the pixels outside the bed area. For example, the values of pixels outside of the bed can be multiplied by 0.5 while the value of the pixels inside the of the bed can remain the same (e.g., multiplied by 1). The “in-bed center” of a cluster is the centroid of the sum of the in-bed feature maps for the cluster.

Steps 1501-1504 can be the same as or similar to steps 1401-1404, respectively. The time period in which the FMCW wireless signals are produced in step 1501 includes multiple days.

In step 1505, at least some of the feature maps are automatically grouped into daily clusters, for example in the same or similar manner as in step 650. Each daily cluster includes feature maps from the same day of the time period, though there may be more than one daily cluster for any one day. Each daily cluster has a respective group of correlated breathing signals.

In step 1506, a plurality of anchor clusters, from the daily clusters, are automatically identified. The anchor clusters have respective groups of correlated breathing signals that mutually overlap in time. The daily clusters that are not identified as anchor clusters can be referred to as non-anchor daily clusters.

The anchor clusters likely represent different people because the breathing signals overlap temporally. The duration of each daily cluster can be defined to be the sum of the durations of the breathing signals in a respective daily cluster. The overlap duration between a pair of daily clusters (e.g., first and second daily clusters) can be defined as the overlap in time between the breathing signals in one cluster and those in the other. For example, the overlap ratio can be defined as:

overlap ratio=overlap duration/min(duration of first daily cluster, duration of second daily cluster)

The anchor clusters can be identified as having the pair of daily clusters having the highest overlap ratio. Alternatively, the anchor clusters can be identified as arbitrary pair of daily clusters that has an overlap ratio that is higher than a predetermined threshold value (e.g., 0.6). Alternatively, the anchor clusters can be identified by picking two daily clusters that temporally overlap.

In step 1507, a plurality of meta clusters are automatically formed. Each meta cluster includes one of the anchor clusters identified in step 1506. A meta cluster can represent a group or collection of daily clusters.

In step 1508, at least some of the non-anchor daily clusters are automatically added to the meta clusters according to first statistical correlations between each non-anchor daily cluster and each meta cluster.

For example, for each non-anchor cluster C, a meta cluster that contains another cluster D satisfying one or both of the following two statistical correlations: (i) the magnitude of a statistical correlation, such as a Pearson correlation, between the average feature maps of C and D is above some fixed threshold (e.g., 0.8); and/or (ii) the difference in average breathing rate between C and D is below some fixed threshold (e.g., 2 breaths per minute). For purposes of this statistical correlation, differences in breathing rates are always non-negative values.

For each non-anchor cluster C where there is more than one meta cluster that satisfies the statistical correlations (i) or (ii), then both statistical correlations (i) and (ii) can be performed. Additionally or alternatively, the non-anchor cluster C can be added to the meta cluster in which the statistical correlation(s) in (i) and/or in (ii) is/are the highest (e.g., compared to the other meta cluster(s) that satisfy the statistical correlations (i) and/or (ii)).

For each non-anchor cluster C where there are no meta clusters that satisfy the statistical correlations (i) and/or (ii), a new meta cluster can be formed and the respective non-anchor cluster C can be added to the respective new meta cluster.

In step 1509, at least some of the meta clusters are automatically merged to form merged meta clusters according to second statistical correlations between the meta clusters.

For example, a Pearson correlation be computed for each meta cluster. The absolute value or magnitude of the Pearson correlation can computed such that all Pearson correlations are non-negative. In some embodiments, the 90^{th} percentile Pearson correlation between the average feature maps for pairs of individual clusters—one from M1 and the other from M2—can be computed. The 90^{th} percentile Pearson correlation is compared to a predetermined threshold value such as 0.6.

A pair of meta clusters M1 and M2 can be a candidate pair for merger when the magnitude of (or absolute value of) the 90^{th} percentile Pearson correlation between the average feature maps for pairs of individual clusters is above the predetermined threshold value. A pair of meta clusters M1 and M2 is not considered a candidate pair for merger when the magnitude of (or absolute value of) the 90^{th} percentile Pearson correlation between the average feature maps for pairs of individual clusters is equal to or below the predetermined threshold value. In other embodiments, a pair of meta clusters M1 and M2 can be merged into a merged meta cluster when the magnitude of (or absolute value of) the 90^{th} percentile Pearson correlation between the average feature maps for pairs of individual clusters is above the predetermined threshold value

To do determine whether to merge a candidate pair of meta clusters M1 and M2, a correlation can be calculated. The correlation can be related to the relative size ratios of the candidate pair of meta clusters M1 and M2 and the average breathing rates of the individual clusters in the meta clusters M1 and M2. For example, the correlation between a candidate pair of meta clusters M1 and M2 can be calculated as follows:

$$Correlation = A + \frac{min ( R ( M 1 ) , R ( M 2 ) )}{10} - \frac{B}{3}$$

where A is the average of the Pearson correlation between the average feature maps for pairs of individual clusters, one from M1 and the other from M2; and B is the median of the difference in average breathing rates between all pairs of individual clusters, one from M1 and the other from M2. R(M) is a relative size ratio of a respective meta cluster (e.g., R(M1) and R(M2) are the relative size ratios of meta clusters M1 and M2, respectively). R(M) is equal to the number of clusters in M divided by the maximum number of clusters (e.g., non-anchor clusters and anchor clusters) in any meta cluster. Meta clusters containing a large number of clusters generally have higher relative size ratios R(M). The calculated correlation can be compared to a predetermined threshold (e.g., 0.1). If the calculated correlation is above the predetermined threshold, the candidate pair of meta clusters M1 and M2 are merged into a merged meta cluster. If the calculated correlation is equal to or lower than the predetermined threshold, the candidate pair of meta clusters M1 and M2 are not merged.

The process can be repeated until all candidate pair of meta clusters are processed (e.g., merged or not merged depending on the respective calculated correlations).

In step 1510, respective distances between each merged meta cluster and one or more predetermined sleep areas of one or more respective people in the room are automatically determined.

In one example, for each merged meta cluster, the average of all feature maps in all clusters in a respective merged meta cluster can be calculated to form an average feature map. In the average feature map, the pixel with the highest value is identified.

Next, cover the room with a two-dimensional mesh. For each square in the mesh, the average of the pixels in the square in the average feature map can be computed. Squares whose average values are not greater than half the value of the pixel with the highest value in the average feature map can be excluded from further consideration.

The minimum distance ∂ from any non-excluded square to the sleep area of person 1 can be determined. This distance can be considered negative (e.g., −1) if there is a point in the square that is within the sleep area, and positive otherwise. A score can be computed for each merged meta cluster using the equation:

$$Score = \partial - 0.5 ln ( \frac{duration}{maximum duration} )$$

where ln is the natural logarithm function, duration is the sum of the durations of the breathing signals in all of the clusters in the merged meta cluster, and maximum duration is the maximum duration of any merged meta cluster.

In step 1511, the breathing signals associated with a first merged meta cluster are automatically assigned to a first person based, at least in part, on one or more first respective distances between the first merged meta cluster and each predetermined sleep area.

For example, the breathing signals associated with the merged meta cluster with the lowest score (which may be negative) can be assigned to person 1.

In step 1512, the breathing signals associated with a second merged meta cluster are automatically assigned to a second person based, at least in part, on one or more second respective distances between the second merged meta cluster and each predetermined sleep area.

For example, the breathing signals associated with the merged meta cluster with the second lowest score is assigned to person 2.

In some embodiments, a first minimum distance ∂1 from any non-excluded square to the sleep area of person 1 and a second minimum distance ∂2 from any non-excluded square to the sleep area of person 2 can be calculated. First and second scores can then be calculated respectively as:

$$Score 1 = {\partial }_{1} - 0.5 ln ( \frac{duration}{maximum duration} )$$

$$Score 2 = {\partial }_{2} - 0.5 ln ( \frac{duration}{maximum duration} )$$

The breathing signals associated with the merged meta cluster with the lowest first score (which may be negative) can be assigned to person 1. The breathing signals associated with the merged meta cluster with the lowest second score (which may be negative) can be assigned to person 2.

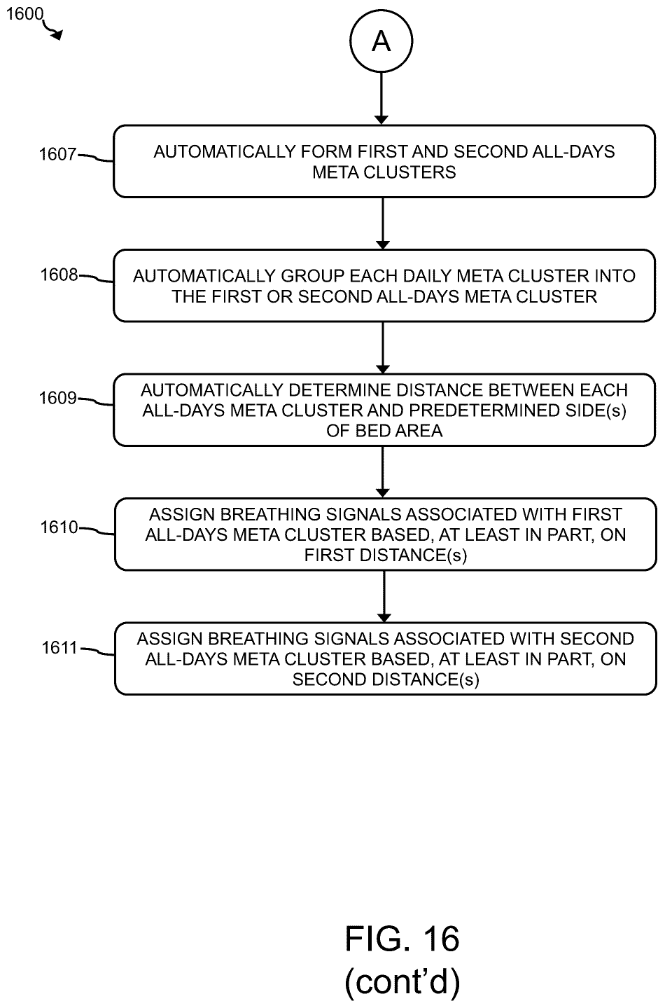

FIG. 16 is a flow chart of a method 1600 for automatically determining the source of breathing signals from multiple people according to another embodiment. Method 1600 can be implemented using motion tracking system(s) 100, 40, and/or 520.

Steps 1601-1605 can be the same as or similar to steps 1501-1405, respectively. The time period in which the FMCW wireless signals are produced in step 1601 includes multiple days.

In step 1606, the daily clusters for a respective day are automatically grouped into one or more daily meta clusters according to first statistical correlations between the daily clusters for the respective day.

Step 1: Goal: Merge clusters from the same day that are identified as the same person.

For each day, a list of daily meta clusters, which is initially empty, can be maintained. Each daily meta cluster comprises a collection of daily clusters from the same day and identified as belonging to the same person, as discussed herein.

For each day, iterate through all daily clusters for the day in order of decreasing duration, i.e., longest first. The duration of a daily cluster is the sum of the durations of the breathing signals in the cluster. The daily clusters can be iterated through in different orders or sequences in other embodiment. The daily cluster considered during an iteration can be referred to as a “current daily cluster.”

Add the current daily cluster to a daily meta cluster if:
(a) the average correlation between the feature maps in the current cluster and those in the daily meta cluster is above a fixed threshold (e.g., above 0.2 if the distance between their in-bed centers is less than 0.4 m, and above 0.3 otherwise); and/or
(b) the average breathing rates of the candidate breathing signals in the current cluster and the daily meta cluster do not differ a predetermined number (e.g., by less than or equal to 2 breaths per minute); and/or
(c) the candidate breathing signals in the current cluster and the daily meta cluster are lower than or equal to a maximum overlap ratio (e.g., 0.4), where the overlap ratio is defined as the overlap duration divided by max (two hours, min (duration of current cluster, duration of daily meta cluster)).

If more than one daily meta cluster satisfies conditions (a), (b), and (c), the current cluster can be added to the daily meta cluster that has the smallest overlap ratio.

If no daily meta cluster satisfies conditions (a), (b), and (c), a new daily meta cluster can be created that includes the current cluster.

Step 2: Goal: Remove daily meta clusters so that at most three remain per day.

For each day in the time period, remove a daily meta cluster if:
(a) the weight of the pixels in the vicinity of the pixel with the largest value in a daily meta cluster's average in-bed feature map is too small. The weight can be calculated as follows. First, all pixel values in each daily meta cluster's average feature map are normalized by dividing by the value of the largest value pixel. Then create an in-bed feature map using the normalized average feature map. An in-bed feature map can be calculated by weighting the values of the normalized average feature map pixels in the bed area more heavily than the pixels outside the bed area. For example, the values of pixels outside of the bed can be multiplied by 0.5 while the value of the pixels inside the of the bed can remain the same (e.g., multiplied by 1). Then the values of the pixels in a 5-by-5 grid centered on the largest value pixel are summed up. If this sum is less than a certain threshold (e.g., less than 2), then the weight is considered too small, as in this case the daily meta cluster is not likely to come from the bed area; and/or
(b) the total duration of the daily meta cluster is smaller than a predetermined value (e.g., less than 1 hour if it has the largest duration, less than 1.5 hours if it has the second largest duration, and less than 2 hours if it has the third largest duration); and/or
(c) the total duration of the daily meta cluster is fourth largest or smaller.

Step 3: Goal: Adjust the bed location if it appears to be inaccurate.

If the total number of daily meta clusters, across all days in the time window, is less than one third of the total number of days in the time window, adjust the center of the bed to be the centroid of the sum of all feature maps in all clusters from all days in the time window. The size of the bed and the sides on which people sleep are not changed. If the center of the bed has changed, go back to Step 1 and start over.

Step 4: Goal: Reduce the maximum number of daily meta clusters for any day in the time period from three to two.

If, for any day in the time window, there are three daily meta clusters, remove a daily meta cluster if:
(a) There are at least five days in the time window and the daily meta cluster has the lowest average correlation score with all other daily meta clusters in the time window. The correlation between two daily meta clusters can be the average correlation between the feature maps in the two daily meta clusters; or
(b) There are fewer than five days in the time window, and the daily meta cluster has the shortest total duration among the three daily meta clusters for that day.

In step 1607, first and second all-days meta clusters are automatically formed.

In step 1608, each daily meta cluster is automatically grouped into first or second all-days meta cluster.

Example implementations of steps 1607 and 1608 are described below.

Step 5: Goal: Determine whether one person or two people sleep in the room at the same time, and handle with separate cases.

If there is at least one day with two daily meta clusters, continue to step 6. Otherwise, go to step 7.

Step 6: Goal: Put daily meta clusters from days with two daily meta clusters into two all-days meta clusters A1 and A2.

Find a day with two daily meta clusters D1 and D2, and put D1 into all-days meta cluster A1, and put D2 into all-days meta cluster A2.

Iterate through all days in the time window. For each day with two daily meta clusters D1 and D2, put D1 into A1 and D2 into A2 if:

$$similarity ( A 1 , D 1 ) + similarity ( A 2 , D 2 ) \geq similarity ( A 1 , D 2 ) + similarity ( A 2 , D 1 )$$

where similarity (A,D) is equal to the average correlation between the feature maps in A and D divided by a function f(difference in average breathing rate of A and D) where function f is a step function that applies a penalty if the difference in breathing rate is too large. If the inequality does not hold, then put D1 into A2 and put D2 into A1.

Step 7: Goal: Create all-days meta clusters and assign identities in the case when only one person is present on any given day.
(a) Select an arbitrary daily meta cluster D and put it into a new all-days meta cluster A1′.
(b) Put all daily meta clusters D′ where similarity (D′, A1′) is above a certain threshold into A1′, where the similarity is determined as described above in step 6.
(c) If there are any remaining daily meta clusters, repeat steps 7(a) and 7(b) to form another all-days meta cluster. Continue until no daily meta clusters remain.
(d) Let A1 and A2 be the all-days meta clusters with the largest duration. Remove daily meta clusters from all other all-days meta clusters. Put any such daily meta cluster D into A1 if:
similarity (D, A1)≥similarity (D, A2),
otherwise put D into A2.

In step 1609, the respective distance between each all-days meta cluster and one or more predetermined side(s) of a bed area is automatically determined.

The distance between a respective in-bed center location of each all-days meta cluster and a first predetermined side of the bed area assigned to person 1 is determined. The distance between the respective in-bed center location of each all-days meta cluster and a second predetermined side of the bed area assigned to person 2 can also be determined.

In step 1610, the breathing signals associated with the first all-days meta cluster are automatically assigned based, at least in part, one or more first respective distances between a first in-bed center location of the first all-days meta cluster and each predetermined side of the bed area.

In step 1611, the breathing signals associated with the second all-days meta cluster are automatically assigned based, at least in part, one or more second respective distances between a second in-bed center location of the second all-days meta cluster and each predetermined side of the bed area.

Example implementations of steps 1610 and 1611 are described below.

When the distances determined in step 1609 is only with respect to the first predetermined side of the bed area assigned to person 1, the breathing signals associated with the all-days meta cluster A1 or A2 having the smallest distance are assigned to person 1 and the breathing signals associated with other all-days meta cluster A2 or A1 (having the largest distance) are assigned to person 2.

When the distances determined in step 1609 is with respect to the first predetermined side of the bed area assigned to person 1 and with respect to the second predetermined side of the bed area assigned to person 2, the breathing signals associated with the all-days meta cluster A1 or A2 having the smallest distance to the first predetermined side of the bed area are assigned to person 1 and the breathing signals associated with the all-days meta cluster A2 or A1 having the smallest distance to the second predetermined side of the bed area are assigned to person 1.

Additionally or alternatively, the breathing signals associated with each all-days meta cluster A1 or A2 can also be assigned based, at least in part, on the following:
(a) If the similarity score between A1 and A2 is above a fixed threshold and the overlap is below a fixed threshold, both A1 and A2 can be assigned to person 1; and/or
(b) If the duration of the all-days cluster (A1 or A2) assigned to person 2 is greater than twice the duration of the cluster assigned to person 1, the assignments of A1 and A2 can be swapped.

In some embodiments, the breathing signal data from prior days in the time period can be used to determine the source of breathing signals from a current or most-recent day. For example, for each candidate breathing signal in the current day in the time period, find the daily meta-cluster that contains the breathing signal's feature map and output the identity of the person to which the daily meta-cluster has been assigned.

The invention should not be considered limited to the particular embodiments described above. Various modifications, equivalent processes, as well as numerous structures to which the invention may be applicable, will be readily apparent to those skilled in the art to which the invention is directed upon review of this disclosure. The above-described embodiments may be implemented in numerous ways. One or more aspects and embodiments involving the performance of processes or methods may utilize program instructions executable by a device (e.g., a computer, a processor, or other device) to perform, or control performance of, the processes or methods.

In this respect, various inventive concepts may be embodied as a non-transitory computer readable storage medium (or multiple non-transitory computer readable storage media) (e.g., a computer memory of any suitable type including transitory or non-transitory digital storage units, circuit configurations in Field Programmable Gate Arrays or other semiconductor devices, or other tangible computer storage medium) encoded with one or more programs that, when executed on one or more computers or other processors, perform methods that implement one or more of the various embodiments described above. When implemented in software (e.g., as an app), the software code may be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more communication devices, which may be used to interconnect the computer to one or more other devices and/or systems, such as, for example, one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Also, a computer may have one or more input devices and/or one or more output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

The non-transitory computer readable medium or media may be transportable, such that the program or programs stored thereon may be loaded onto one or more different computers or other processors to implement various one or more of the aspects described above. In some embodiments, computer readable media may be non-transitory media.

The terms “program,” “app,” and “software” are used herein in a generic sense to refer to any type of computer code or set of computer-executable instructions that may be employed to program a computer or other processor to implement various aspects as described above. Additionally, it should be appreciated that, according to one aspect, one or more computer programs that when executed perform methods of this application need not reside on a single computer or processor but may be distributed in a modular fashion among a number of different computers or processors to implement various aspects of this application.

Computer-executable instructions may be in many forms, such as program modules, executed by one or more computers or other devices. Generally, program modules include routines, programs, objects, components, data structures, etc. that performs particular tasks or implement particular abstract data types. The functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Thus, the disclosure and claims include new and novel improvements to existing methods and technologies, which were not previously known nor implemented to achieve the useful results described above. Users of the method and system will reap tangible benefits from the functions now made possible on account of the specific modifications described herein causing the effects in the system and its outputs to its users. It is expected that significantly improved operations can be achieved upon implementation of the claimed invention, using the technical components recited herein.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. A system for wireless detection of breathing from multiple people, comprising:
one or more transmitting antennas configured to produce transmitted wireless signals over a time period;
one or more transmitting one or more receiving antennas configured to receive reflected wireless signals over the time period, the reflected wireless signals being reflected from objects and/or people in a room; and
a computer in electrical communication with the one or more receiving antennas to receive the reflected wireless signals, the computer including a processor and non-transitory computer memory in electrical communication with the processor, the non-transitory computer memory storing computer-readable instructions that, when executed by the processor, cause the processor to:
automatically extract breathing signals from the reflected wireless signals;
automatically form feature maps for the breathing signals, each feature map representing a correlation of the breathing signals at each voxel in a plurality of voxels that represent three-dimensional locations within the room, each feature map representing a respective time interval of the time period, the time period subdivided into time intervals;
automatically statistically compare the features maps with one or more location templates, each location template including a respective correlation of collected-breathing signals for a respective known person at each voxel; and
automatically identify the breathing signals for a first person and the breathing signals for a second person based, at least in part, on statistical correlations between each location template and each feature map.

2. The system of claim 1, wherein:
the one or more location templates comprise:
a first location template that includes a first correlation of the collected-breathing signals for the first person at each voxel, and
a second location template that includes a second correlation of the collected breathing signals for a second person at each voxel, and
the computer-readable instructions further cause the processor to:
automatically assign, for each feature map having a higher statistical correlation with the first location template compared to with the second location template, the breathing signals associated with a respective feature map to the first person; and
automatically assign, for each feature map having a lower statistical correlation with the first location template compared to with the second location template, the breathing signals associated with the respective feature map to the second person.

3. The system of claim 1, wherein the statistical correlations include Pearson correlations.

4. The system of claim 1, wherein the computer-readable instructions further cause the processor to:
automatically calculate a respective average breathing rate of the breathing signals associated with each feature map; and
automatically statistically compare the respective average breathing rate to a respective average location-template breathing rate for each location template.

5. The system of claim 4, wherein the statistical correlations include:
a first statistical comparison of the respective average breathing rate for each feature map with a first average location-template breathing rate of the first location template, and
a second statistical comparison of the respective average breathing rate for each feature map with a second average location-template breathing rate of the second location template.

6. The system of claim 1, wherein:
the one or more location templates comprise a first location template that includes a first correlation of the collected-breathing signals for the first person at each voxel, and
the computer-readable instructions further cause the processor to:
automatically assign, for each feature map having a respective statistical correlation with the first location template higher than a threshold value, the breathing signals associated with a respective feature map to the first person; and
automatically assign, for each feature map having the respective statistical correlation with the first location template lower than or equal to the threshold value, the breathing signals associated with the respective feature map to the second person.

7. The system of claim 1, wherein the computer-readable instructions further cause the processor to:
automatically group at least some of the feature maps into clusters, each cluster representing a group of correlated breathing signals; and
automatically identify the breathing signals for the first person and the breathing signals for a second person based, at least in part, on statistical correlations between each location template and the feature maps in each cluster.

8. The system of claim 1, wherein:
the time period is a second time period,
the one or more transmitting antennas are configured to produce first transmitted wireless signals over a first time period, the first time period before the second time period
the one or more receiving antennas are configured to receive first reflected wireless signals over the first time period, the first reflected wireless signals being reflected from the objects and/or the people in the room, and
the computer-readable instructions further cause the processor to:
automatically extract the collected breathing signals, with the computer, from the first reflected wireless signals;
automatically form first feature maps for the first breathing signals, each first feature map representing a first correlation of the first breathing signals at each voxel, each first feature map representing a respective first time interval of the first time period, the first time period subdivided into first time intervals;
automatically group at least some of the first feature maps into one or more clusters, each cluster representing a group of correlated first breathing signals;
automatically form the one or more location templates using the one or more clusters; and
label each location template with the respective known person.

9. A system for wireless detection of breathing from multiple people, comprising:
one or more transmitting antennas configured to produce transmitted wireless signals over a time period that includes multiple days;
one or more transmitting one or more receiving antennas configured to receive reflected wireless signals over the time period, the reflected wireless signals being reflected from objects and/or people in a room; and
a computer in electrical communication with the one or more receiving antennas to receive the reflected wireless signals, the computer including a processor and non-transitory computer memory in electrical communication with the processor, the non-transitory computer memory storing computer-readable instructions that, when executed by the processor, cause the processor to:
a. automatically extract breathing signals from the reflected wireless signals;
b. automatically form feature maps for the breathing signals, each feature map representing a correlation of the breathing signals at each voxel in a plurality of voxels that represent three-dimensional locations within the room, each feature map representing a respective time interval of the time period, the time period subdivided into time intervals;
c. automatically group at least some of the feature maps into daily clusters, each daily cluster representing a group of correlated breathing signals for one of the days of the time period;
d. automatically identify a plurality of anchor clusters, from the daily clusters, having respective groups of the correlated breathing signals that mutually overlap in time;
e. automatically form a plurality of meta clusters, each meta cluster including a respective anchor cluster, wherein the daily clusters not including the anchor clusters comprise non-anchor daily clusters;
f. automatically add at least some of the non-anchor daily clusters to the meta clusters according to first statistical correlations between each non-anchor daily cluster and each meta cluster;
g. automatically merge at least some of the meta clusters to form merged meta clusters according to second statistical correlations between the meta clusters;
h. automatically determine respective distances between each merged meta cluster and one or more predetermined sleep areas of one or more respective people in the room;
i. automatically assign the breathing signals associated with a first merged meta cluster to a first person based, at least in part, on one or more first respective distances between the first merged meta cluster and each predetermined sleep area; and
j. automatically assign the breathing signals associated with a second merged meta cluster to a second person based, at least in part, on one or more second respective distances between the second merged meta cluster and each predetermined sleep area.

10. The system of claim 9, wherein for each respective non-anchor daily cluster not added to one of the meta clusters in step f, a respective new meta cluster is formed that includes the respective daily cluster.

11. The system of claim 9, wherein the first statistical correlations include:
a Pearson correlation between average feature maps, and/or
a difference between average breathing rates.

12. The system of claim 9, wherein the second statistical correlations include:
a Pearson correlation between average feature maps, and/or
a difference between average breathing rates.

13. The system of claim 9, wherein:
the one or more predetermined sleep areas comprise a first predetermined sleep area for a first person in the room, and
the computer-readable instructions further cause the processor to:
automatically assign the breathing signals associated with the first merged meta cluster to the first person based on a first distance between the first merged meta cluster and the first predetermined sleep area; and
automatically assign the breathing signals associated with the second merged meta cluster to the second person based on a second distance between the first merged meta cluster and the first predetermined sleep area, the first distance smaller than the second distance.

14. The system of claim 9, wherein:
the one or more predetermined sleep areas comprise:
a first predetermined sleep area for a first person in the room, and
a second predetermined sleep area for a second person in the room, and
the computer-readable instructions further cause the processor to:
automatically determine first distances between each merged meta cluster and the first predetermined sleep area;
automatically determine second distances between each merged meta cluster and the second predetermined sleep area;
automatically assign the breathing signals associated with the first merged meta cluster to the first person, the first merged meta cluster having a smallest first distance with respect to the first distances, and
automatically assign the breathing signals associated with the second merged meta cluster to the second person, the second merged meta cluster having a smallest second distance with respect to the second distances.

15. A system for wireless detection of breathing from multiple people, comprising:
one or more transmitting antennas configured to produce transmitted wireless signals over a time period that includes multiple days;
one or more transmitting one or more receiving antennas configured to receive reflected wireless signals over the time period, the reflected wireless signals being reflected from objects and/or people in a room; and
a computer in electrical communication with the one or more receiving antennas to receive the reflected wireless signals, the computer including a processor and non-transitory computer memory in electrical communication with the processor, the non-transitory computer memory storing computer-readable instructions that, when executed by the processor, cause the processor to:
a. automatically extract breathing signals from the reflected wireless signals;
b. automatically form feature maps for the breathing signals, each feature map representing a correlation of the breathing signals at each voxel in a plurality of voxels that represent three-dimensional locations within the room, each feature map representing a respective time interval of the time period, the time period subdivided into time intervals;
c. automatically group at least some of the feature maps into daily clusters, each daily cluster representing a group of correlated breathing signals for one of the days of the time period;
d. automatically the daily clusters for a respective day into one or more daily meta clusters according to first statistical correlations between the daily clusters for the respective day;
e. automatically form first and second all-days meta clusters, the first all-days meta cluster including a first daily meta cluster, the second all-days meta cluster including a second daily meta cluster;
f. automatically group each daily meta cluster for each day into the first all-days meta cluster or the second all-days meta cluster, the grouping determined based, at least in part, on second statistical correlations between each daily meta cluster and each all-days meta cluster;
g. automatically determine a respective distance between a respective in-bed center location of each all-days meta cluster and one or more predetermined sides of a bed area of one or more respective people in the room;
h. automatically assign the breathing signals associated with the first all-days meta cluster to a first person based, at least in part, on one or more first respective distances between a first in-bed center location of the first all-days meta cluster and each predetermined side of the bed area; and
i. automatically assign the breathing signals associated with the second all-days meta cluster to a second person based, at least in part, on one or more first respective distances between a second in-bed center location of the second all-days meta cluster and each predetermined side of the bed area.

16. The system of claim 15, wherein:
the one or more predetermined sides of the bed area comprises a first predetermined side of the bed area for the first person, and
the computer-readable instructions further cause the processor to:
automatically assign the breathing signals associated with the first all-days meta cluster to the first person based, at least in part, on a first distance between the first in-bed center location and the first predetermined side of the bed area; and
automatically assign the breathing signals associated with the second all-days meta cluster to the second person based, at least in part, on a second distance between the second in-bed center location and the first predetermined side of the bed area, the first distance smaller than the second distance.

17. The system of claim 15, wherein:
the one or more predetermined sides of the bed area comprises:
a first predetermined side of the bed area for the first person, and
a second predetermined side of the bed area for the second person, and
the computer-readable instructions further cause the processor to:
automatically determine first distances between each in-bed center location and the first predetermined sleep area;
automatically determine second distances between each in-bed center location and the second predetermined sleep area;
automatically assign the breathing signals associated with the first all-days meta cluster to the first person, the first in-bed center location having a smallest first distance with respect to the first distances, and
automatically assign the breathing signals associated with the second all-days meta cluster to the second person, the second in-bed center location having a smallest second distance with respect to the second distances.

18. The system of claim 15, wherein:
the first person is a primary person under observation,
in steps h and i, the first all-days meta cluster is assigned to the first person and the second all-days meta cluster is assigned to the second person, and
the computer-readable instructions further cause the processor to:
determine a first duration of the breathing signals associated with the first all-days meta cluster and a second duration of the breathing signals associated with the second all-days meta cluster; and
after step i:
automatically reassign the first all-days meta cluster to the second person; and
automatically reassign the second all-days meta cluster to the first person second, wherein the first duration is greater than twice the second duration.

19. The system of claim 15, wherein:
the daily clusters from a first day are grouped into only the first daily meta cluster for the first day,
the first all-days meta cluster is formed with the first daily meta cluster from the first day, and
the second all-days meta cluster is formed with the second daily meta cluster from a second day, wherein a respective second statistical correlation between the second daily meta cluster from the second day and the first all-days meta cluster is lower than or equal to a threshold value.

20. The system of claim 15, wherein:
the first all-days meta cluster is formed with the first daily meta cluster from a first day, and
the second all-days meta cluster is formed with the second daily meta cluster from the first day.
